SEQUENCE LISTING (43 sequences)

SEQ ID NO: 1 - protein, 24 aa, synthetic construct
  VARIANT 6: note = X is K, A, L, N or C
  VARIANT 7: note = X is K or L
  VARIANT 11: note = X is S, K, or R
  VARIANT 13: note = X is I, R, or K
  VARIANT 15: note = X is K or R
  VARIANT 16: note = X is K or R
  VARIANT 19: note = X is K, R, or E
  VARIANT 20: note = X is K or R
  VARIANT 21: note = X is M or I
  VARIANT 22: note = X is L, R, or K
  VARIANT 23: note = X is N, R, or K
  VARIANT 24: note = X is S or I
NGRKAXXNPAXPXVXXIIXXXXXX

SEQ ID NO: 2 - protein, 24 aa, synthetic construct
NGRKACLNPASPIVKKIIEKMLNS

SEQ ID NO: 3 - protein, 24 aa, synthetic construct
NGRKACLNPASPIVKKIIKKMLNS

SEQ ID NO: 4 - protein, 24 aa, synthetic construct
NGRKACLNPASPIVKKIIKKILNS

SEQ ID NO: 5 - protein, 24 aa, synthetic construct
NGRKACLNPASPIVKKIIKKILKS

SEQ ID NO: 6 - protein, 24 aa, synthetic construct
NGRKACLNPASPIVKKIIKKILKI

SEQ ID NO: 7 - protein, 24 aa, synthetic construct
NGRKACLNPASPKVKKIIKKIKKI

SEQ ID NO: 8 - protein, 23 aa, synthetic construct
NGRKACLNPASIVKKIIKKILNS

SEQ ID NO: 9 - protein, 23 aa, synthetic construct
NGRKACLNPASIVKKIIKKILKS

SEQ ID NO: 10 - protein, 23 aa, synthetic construct
NGRKACLNPASIVKKIIKKILKI

SEQ ID NO: 11 - protein, 23 aa, synthetic construct
NGRKACLNPASKVKKIIKKIKKI

SEQ ID NO: 12 - protein, 23 aa, synthetic construct
NGRKACLNPAKKVKKIIKKIKKI

SEQ ID NO: 13 - protein, 24 aa, synthetic construct
NGRKAKKNPASPIVKKIIEKMLNS

SEQ ID NO: 14 - protein, 24 aa, synthetic construct
NGRKAKKNPASPIVKKIIKKILKI

SEQ ID NO: 15 - protein, 24 aa, synthetic construct
NGRKAKKNPASPKVKKIIKKIKKI

SEQ ID NO: 16 - protein, 23 aa, synthetic construct
NGRKAKKNPASIVKKIIKKILKI

SEQ ID NO: 17 - protein, 23 aa, synthetic construct
NGRKAKKNPASKVKKIIKKIKKI

SEQ ID NO: 18 - protein, 23 aa, synthetic construct
NGRKAKKNPAKKVKKIIKKIKKI

SEQ ID NO: 19 - protein, 22 aa, synthetic construct
NGRKACLNASIVKKIIKKILNS

SEQ ID NO: 20 - protein, 22 aa, synthetic construct
NGRKACLNAKKVKKIIKKIKKI

SEQ ID NO: 21 - protein, 22 aa, synthetic construct
NGRKAKKNAKKVKKIIKKIKKI

SEQ ID NO: 22 - protein, 24 aa, synthetic construct
NGRKACLNPASPIVRRIIERMLNS

SEQ ID NO: 23 - protein, 24 aa, synthetic construct
NGRKACLNPASPIVRRIIRRILRI

SEQ ID NO: 24 - protein, 23 aa, synthetic construct
NGRKACLNPARRVRRIIRRIRRI

SEQ ID NO: 25 - protein, 23 aa, synthetic construct
NGRKAKKNPARRVRRIIRRIRRI

SEQ ID NO: 26 - protein, 23 aa, synthetic construct
NGRKAKLNPASIVKKIIKKILKS

SEQ ID NO: 27 - protein, 23 aa, synthetic construct
NGRKAALNPASIVKKIIKKILKS

SEQ ID NO: 28 - protein, 23 aa, synthetic construct
NGRKALLNPASIVKKIIKKILKS

SEQ ID NO: 29 - protein, 23 aa, synthetic construct
NGRKANLNPASIVKKIIKKILKS

SEQ ID NO: 30 - protein, 23 aa, synthetic construct
NGRKAKLNPASFFKKIIKKILKS

SEQ ID NO: 31 - protein, 23 aa, synthetic construct
NGRKAKLNPASIVKKFFKKILKS

SEQ ID NO: 32 - protein, 23 aa, synthetic construct
NGRKAKLNPASIVKKIIKKFFKS

SEQ ID NO: 33 - protein, 23 aa, synthetic construct
NGRKAKLNPASFFKKFFKKILKS

SEQ ID NO: 34 - protein, 23 aa, synthetic construct
NGRKAKLNPASIVKKFFKKFFKS

SEQ ID NO: 35 - protein, 23 aa, synthetic construct
NGRKAKLNPASFFKKIIKKFFKS

SEQ ID NO: 36 - protein, 23 aa, synthetic construct
NGRKAKLNPASFFKKFFKKFFKS

SEQ ID NO: 37 - protein, 14 aa, synthetic construct
ASIVKKIIKKILKS

SEQ ID NO: 38 - protein, 21 aa, synthetic construct
RKAKLNPASIVKKIIKKILKS

SEQ ID NO: 39 - protein, 19 aa, synthetic construct
AKLNPASIVKKIIKKILKS

SEQ ID NO: 40 - protein, 17 aa, synthetic construct
LNPASIVKKIIKKILKS

SEQ ID NO: 41 - protein, 24 aa, synthetic construct
  VARIANT 6: note = X is K, A, L, N, or C
  VARIANT 7: note = X is K or L
  VARIANT 11: note = X is S, K, or R
  VARIANT 13: note = X is I, R, K, or F
  VARIANT 14: note = X is K or R
  VARIANT 15: note = X is K or R
  VARIANT 17: note = X is K or R
  VARIANT 16: note = X is K, R, or E
  VARIANT 18: note = X is M, I, or F
  VARIANT 19: note = X is L, R, K, or F
  VARIANT 20: note = X is N, R, or K
  VARIANT 21: note = X is S or I
  VARIANT 22: note = X is V or F
  VARIANT 23: note = X is I or F
  VARIANT 24: note = X is I or F
NGRKAXXNPAXPXXXXXXXXXXXX

SEQ ID NO: 42 - protein, 12 aa, synthetic construct
IVKKIIEKMLNS

SEQ ID NO: 43 - protein, 12 aa, synthetic construct
NGRKACLNPASP

(19) United States
(12) Patent Application Publication
(10) Pub. No.: US 2026/0356901 A1
(43) Pub. Date: Nov. 12, 2026

(54) ONCOLYTIC PEPTIDES

(71) Applicant: The Johns Hopkins University, Baltimore, MD (US)

(72) Inventors: Natsuki Furukawa, Baltimore, MD (US); Aleksander S. Popel, Baltimore, MD (US); Niranjan B. Pandey, Baltimore, MD (US); Adam Mirando, Baltimore, MD (US)

(21) Appl. No.: 19/486,830

(22) PCT Filed: May 23, 2024
(86) PCT No.: PCT/US2024/030812

§ 371 Date: Nov. 21, 2025

Related U.S. Application Data
(60) Provisional application No. 63/503,800, filed on May 23, 2023.
(60) Provisional application No. 63/551,745, filed on Feb. 9, 2024.

(51) Int. Cl. C07K 14/52; A61K 38/00; A61P 35/00

(52) U.S. Cl. CPC C07K 14/52; A61P 35/00; A61K 38/00

(57) ABSTRACT

The present disclosure provides oncolytic peptides with an amino acid sequence of NGRKAX₁X₂NPAX₃PX₄VX₅X₆IIX₇X₈X₉X₁₀X₁₁X₁₂ (SEQ ID NO: 1), wherein X₁ is K, A, L, N or C; X₂ is K or L; X₃ is S, K, or R; X₄ is I, R or K; X₅, X₆, X₈ are each independently K or R; X₇ is K, R, or E; X₉ is M or I; X₁₀ is L, R, or K; X₁₁ is N, R, or K; X₁₂ is S or I, and compositions and methods of use thereof.

### CROSS-REFERENCE TO RELATED APPLICATIONS

This application claims the benefit of U.S. Provisional Application Nos. 63/503,800, filed May 23, 2023, and 63/551,745, filed Feb. 9, 2024, the contents of which are herein incorporated by reference in their entirety.

### FIELD

The present disclosure provides peptides, and compositions and methods of use thereof.

### SEQUENCE LISTING STATEMENT

The content of the electronic sequence listing titled JHU_41947_601_SequenceListing.xml (Size: 48,782 bytes; and Date of Creation: May 17, 2024) is herein incorporated by reference in its entirety.

### BACKGROUND

Cancer comprises a collection of diseases caused by excessive proliferation of cells in the body that cannot be effectively regulated. Cancer treatment strategies are strongly dependent on several factors including type and stage of cancer, general health considerations, and preferences for the risk/benefits of an individual treatment. Currently, the most widely used strategies to treat cancer include radiotherapy, chemotherapy, immunotherapy, and surgery. The goal of surgery is to remove the cancer or as much of the cancer as possible, and generally, is more effective therapy for early or mid-stage cancers with solid tumors. However, any kind of surgery leads to trauma, bleeding, infection, weakened immunity and other risks. Radiation therapy, including external beam radiation and brachytherapy, is oftentimes used when a patient did not benefit from or has a cancer inaccessible by surgical methods. Radiation therapy is expensive, the course of treatment lasts for an extended period of time and often results in a series of, possibly severe, complications. Chemotherapy broadly describes systemic therapies using pharmaceutical(s) designed to kill fast-growing cells, e.g., cancer cells. However, the ability of these drugs to destroy normal, healthy cells causing side effects, some which may be lifelong. Immunotherapy modulates the body's own immune system to facilitate an anti-tumor effect. However, the benefits can vary widely between individuals and types of cancers and may lead to autoimmune disorders. Overall, current cancer treatment strategies each have an individual set of risks, benefits, and usefulness. Additional therapeutic methods or anti-cancer pharmaceuticals are needed to expand cancer treatment options.

### SUMMARY

Disclosed herein are peptides, or pharmaceutically acceptable salts or solvates thereof, comprising an amino acid sequence of NGRKAX₁X₂NPAX₃PX₄ X₁₃X₅X₆ X₁₄X₁₅X₇X₈X₉X₁₀X₁₁X₁₂ (SEQ ID NO: 41), wherein: X₁ is K, A, L, N or C; X₂ is K or L; X₃ is S, K, or R; X₄ is I, R, K or F; X₅, X₆, X₈ are each independently K or R; X₇ is K, R, or E; X₉ is M, I, or F; X₁₀ is L, R, K, or F; X₁₁ is N, R, or K; X₁₂ is S or I; X₁₃ is V or F; X₁₄ and X₁₅ are each independently I or F; and wherein the peptide has one or more amino acid substitutions, additions or deletions compared to NGRKACLNPASPIVKKIIEKMLNS (SEQ ID NO: 2).

In some embodiments, X₄ and X₁₃ are F. In some embodiments, X₄ is I and X₁₃ is V.

In some embodiments, X₉ and X₁₀ are F. In some embodiments, X₉ is I and X₁₀ is L.

In some embodiments, X₁₄ and X₁₅ are I. In some embodiments, X₁₄ and X₁₅ are F.

In some embodiments, X₅, X₆, X₇, and X₈ are K. In some embodiments, X₁₁ is K and X₁₂ is S. In some embodiments, X₁ is K and X₂ is L. In some embodiments, X₃ is S. In some embodiments, the proline at position 12 is absent.

In some embodiments, the peptide comprises any amino acid sequence of NGRKAX₁X₂NPAX₃PX₄VX₅X₆IIX₇X₈X₉X₁₀X₁₁X₁₂ (SEQ ID NO: 1), wherein: X₁ is K, A, L, N or C; X₂ is K or L; X₃ is S, K, or R; X₄ is I, R or K; X₅, X₆, X₈ are each independently K or R; X₇ is K, R, or E; X₉ is M or I; X₁₀ is L, R, or K; X₁₁ is N, R, or K; and X₁₂ is S or I. In some embodiments, the peptide has one or more amino acid substitutions, additions or deletions compared to the amino acid sequence NGRKACLNPASPIVKKIIEKMLNS (SEQ ID NO: 2).

In some embodiments, X₁ is C and X₂ is L. In some embodiments, X₁ and X₂ are K.

In some embodiments, wherein X₃ is S. In some embodiments, wherein X₉ is I.

In some embodiments, wherein X₁₂ is I. In some embodiments, X₁₁ is K and X₁₂ is I. In some embodiments, X₁₀ is K.

In some embodiments, X₅, X₆, and X₈ are R. In some embodiments, X₇ and X₁₁ are R. In some embodiments, X₉ and X₁₂ are I. In some embodiments, X₁₀ is R. In some embodiments, X₃ and X₄ are R.

In some embodiments, X₅, X₆, and X₈ are K. In some embodiments, X₇ is K. In some embodiments, X₉ is I and X₁₀ is L. In some embodiments, X₁₁ is K and X₁₂ is I.

In some embodiments, the proline at position 9, the proline at position 12, or a combination thereof is absent.

In some embodiments, X₅, X₆, X₇, and X₈ are K. In some embodiments, X₉ is I. In some embodiments, X₁ is C and X₂ is L. In some embodiments, X₁ is K, A, L, or N and X₂ is L. In some embodiments, X₁ and X₂ are K.

In some embodiments, X₃ is S and X₄ is I. In some embodiments, X₁₀ is L, X₁₁ is N or K, and X₁₂ is S or I.

In some embodiments, X₃ and X₄ are K. In some embodiments, X₁₀ and X₁₁ are K and X₁₂ is I.

In some embodiments, the prolines at positions 9 and 12 are absent and X₁ is C and X₂ is L. In some embodiments, X₃ is S or A and X₄ is K or I. In some embodiments, X₁₀ and X₁₁ are K, and X₁₂ is I. In some embodiments, X₁₀ is L, X₁₁ is N, and X₁₂ is S.

In some embodiments, the amino acid sequence is alpha helical.

In some embodiments, the peptide has one or more amino acid substitutions, additions, or deletions compared to NGRKACLNPASIVKKIIKKILKS (SEQ ID NO: 9).

In some embodiments, the peptide comprises one of the following amino acid sequences:

(SEQ ID NO: 3)
NGRKACLNPASPIVKKIIKKMLNS;
(SEQ ID NO: 4)
NGRKACLNPASPIVKKIIKKILNS;
(SEQ ID NO: 5)
NGRKACLNPASPIVKKIIKKILKS;
(SEQ ID NO: 6)
NGRKACLNPASPIVKKIIKKILKI;
(SEQ ID NO: 7)
NGRKACLNPASPKVKKIIKKIKKI;
(SEQ ID NO: 8)
NGRKACLNPASIVKKIIKKILNS;
(SEQ ID NO: 9)
NGRKACLNPASIVKKIIKKILKS;
(SEQ ID NO: 10)
NGRKACLNPASIVKKIIKKILKI;
(SEQ ID NO: 11)
NGRKACLNPASKVKKIIKKIKKI;
(SEQ ID NO: 12)
NGRKACLNPAKKVKKIIKKIKKI;
(SEQ ID NO: 13)
NGRKAKKNPASPIVKKIIEKMLNS;
(SEQ ID NO: 14)
NGRKAKKNPASPIVKKIIKKILKI;
(SEQ ID NO: 15)
NGRKAKKNPASPKVKKIIKKIKKI;
(SEQ ID NO: 16)
NGRKAKKNPASIVKKIIKKILKI;
(SEQ ID NO: 17)
NGRKAKKNPASKVKKIIKKIKKI;
(SEQ ID NO: 18)
NGRKAKKNPAKKVKKIIKKIKKI;
(SEQ ID NO: 19)
NGRKACLNASIVKKIIKKILNS;
(SEQ ID NO: 20)
NGRKACLNAKKVKKIIKKIKKI;
(SEQ ID NO: 21)
NGRKAKKNAKKVKKIIKKIKKI;
(SEQ ID NO: 22)
NGRKACLNPASPIVRRIIERMLNS;
(SEQ ID NO: 23)
NGRKACLNPASPIVRRIIRRILRI;
(SEQ ID NO: 24)
NGRKACLNPARRVRRIIRRIRRI;
(SEQ ID NO: 25)
NGRKAKKNPARRVRRIIRRIRRI;
(SEQ ID NO: 26)
NGRKAKLNPASIVKKIIKKILKS;
(SEQ ID NO: 27)
NGRKAALNPASIVKKIIKKILKS;
(SEQ ID NO: 28)
NGRKALLNPASIVKKIIKKILKS;
(SEQ ID NO: 29)
NGRKANLNPASIVKKIIKKILKS;
(SEQ ID NO: 30)
NGRKAKLNPASFFKKIIKKILKS;
(SEQ ID NO: 31)
NGRKAKLNPASIVKKFFKKILKS;
(SEQ ID NO: 32)
NGRKAKLNPASIVKKIIKKFFKS;
(SEQ ID NO: 33)
NGRKAKLNPASFFKKFFKKILKS;
(SEQ ID NO: 34)
NGRKAKLNPASIVKKFFKKFFKS;
(SEQ ID NO: 35)
NGRKAKLNPASFFKKIIKKFFKS;
and
(SEQ ID NO: 36)
NGRKAKLNPASFFKKFFKKFFKS.

In some embodiments, the peptide comprises an amino acid sequence of ASIVKKIIKKILKS (SEQ ID NO: 37). In some embodiments, the peptide is less than 25 total amino acids.

In some embodiments, the peptide comprises one or more of the following amino acid sequences: NGRKACLNPASIVKKIIKKILKS (SEQ ID NO: 9); NGRKAKLNPASIVKKIIKKILKS (SEQ ID NO: 26); NGRKAALNPASIVKKIIKKILKS (SEQ ID NO: 27); NGRKALLNPASIVKKIIKKILKS (SEQ ID NO: 28); NGRKANLNPASIVKKIIKKILKS (SEQ ID NO: 29); ASIVKKIIKKILKS (SEQ ID NO: 37); RKAKLNPASIVKKIIKKILKS (SEQ ID NO: 38); AKLNPASIVKKIIKKILKS (SEQ ID NO: 39); and LNPASIVKKIIKKILKS (SEQ ID NO: 40).

Also provided are compositions comprising a peptide as disclosed herein, or a nucleic acid encoding thereof. In some embodiments, the compositions further comprise a carrier. In some embodiments, the carrier comprises water and the peptide is at a concentration of 1-100 mg/ml (e.g., 1-50 mg/mL). The peptide concentration may be about 1 mg/mL, about 5 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, or about 100 mg/mL. The peptide concentration may be greater than 5 mg/mL, greater than 10 mg/mL, greater than 20 mg/mL, greater than 30 mg/mL, greater than 40 mg/mL, greater than 50 mg/mL, greater than 60 mg/mL, greater than 70 mg/mL, greater than 80 mg/mL, greater than 90 mg/mL, or greater than 100 mg/mL.

Further disclosed are methods of treating a disease or disorder comprising administering to a subject in need thereof an effective amount of a peptide, or a nucleic acid encoding thereof, or composition disclosed herein. In some embodiments, the disease or disorder comprises cancer. In some embodiments, the disease or disorder comprises breast cancer, skin cancer, and colorectal cancer.

Additionally disclosed are methods for lysing cancer cell membranes and/or inducing cancer cell death comprising contacting a cancer cell with an effective amount of a peptide, or a nucleic acid encoding thereof, or composition disclosed herein. In some embodiments, the cancer cell is in vitro or ex vivo. In some embodiments, the cancer cell is in a subject. In some embodiments, the contacting comprises administering to a subject in need thereof an effective amount of the peptide or a composition thereof.

Other aspects and embodiments of the disclosure will be apparent in light of the following detailed description and accompanying figures.

### BRIEF DESCRIPTION OF THE DRAWINGS

FIG. 1 is a schematic of an exemplary mechanism for oncolytic peptides.

FIGS. 2A-2C show the structure and physical properties of chemokinostatin-1 (CKS1). FIG. 2A is the structure of CKS1 predicted by AlphaFold 2 and its primary amino acid sequence (SEQ ID NO: 2). FIG. 2B shows the electrostatic potential of the surface of CKS1 as predicted by APBS Electrostatics plugin in Pymol. Blue regions are the positively charged regions and the red regions are the negatively charged regions. FIG. 2C shows the hydrophobic regions of CKS1 visualized as red, and the hydrophilic regions are visualized as white.

FIGS. 3A-3H show that CKS1 induces rapid cell death in multiple cancer cell lines but not in non-cancerous cell lines. FIG. 3A is a graph of cell viability of 4T1 murine triple negative breast cancer cells (TNBC) after 100 μM or 20 μM CKS1 treatment measured by real-time cell analyzer (RTCA). Means±SEM, representative of N=3. FIG. 3B is a graph of lactate dehydrogenase (LDH) release in 4T1 cells pre-treated with 50 μM pan-caspase inhibitor Z-VAD for 30 minutes or 50 μM RIPK1 inhibitor Nec-1s for 30 minutes then treated with 100 μM CKS1 for 6 hours. LDH release in the supernatant was measured by the CyQUANT LDH cytotoxicity assay. Means±SEM, N=3, Dunnett's test (***p<0.005). FIGS. 3C-3G are graphs of LDH release in human osteosarcoma U2OS cells, murine fibrosarcoma MCA205 cells, murine colon carcinoma CT26 cells, murine normal NIH/3T3 fibroblasts, and HUVECs, respectively, treated with the indicated concentration of CKS1 for 6 hours. The release of LDH in the supernatant was measured and was normalized to the amount of LDH released from cells treated with lysis buffer. Means±SEM, N=3, Dunnett's test (**p<0.01, ***p<0.005). FIG. 3H is a graph of the incorporation of BrdU in HUVECs treated with CKS1. Means±SEM, N=3, Dunnett's test (**p<0.01, ***p<0.005).

FIGS. 4A-4I show CKS1 induces rupture of the cell membrane in the early time point and apoptosis in the later time point. FIGS. 4A-4D are images of cell morphology of 4T1 cells treated with water (FIGS. 4A and 4B) or 100 μM CKS1 (FIGS. 4C and 4D) for 6 hours. FIGS. 4E-4H are electron microscopy images of the detailed structure of the cellular surface. 4T1 cells had intact cell membrane in the control condition, but the cell membrane was disrupted when they were treated with CKS1 for 30 minutes. When cells were treated with CKS1 for 6 hours, 4T1 cells exhibited an apoptotic morphology. Overall shrinkage of the cell, collapse of the nucleus and chromatin condensation (arrow), and apoptotic blebbing (triangle) indicate that the cell is undergoing apoptosis. FIG. 4I shows the amount of cleaved caspase-3 in the lysate of 4T1 cells treated with CKS1. For the positive control sample, 1 μM staurosporine was applied for 30 minutes. β-tubulin is provided as a loading control.

FIGS. 5A-5C show that the helical region of CKS1 drives the oncolytic activity. FIG. 5A is a graph of the release of LDH in the supernatant measured after treating 4T1 cells with 100 μM of the indicated peptides for 6 hours. Means±SEM, N=3, Dunnett's test (***p<0.005). FIG. 5B is a wheel representation of the helical region of CKS1 and the E19K mutant. Green amino acids represent the hydrophobic amino acids, light blue amino acids represent the neutrally charged amino acids, dark blue amino acids represent the negatively charged amino acids, and the red amino acids represent the positively charged amino acids. FIG. 5C shows the release of LDH in the supernatant was measured after treating 4T1 cells with CKS1 or the E19K mutant at the indicated concentration for 6 hours. Means±SEM, N=3, one-way ANOVA with Tukey-Kramer test.

FIGS. 6A-6D show that CKS1 induces release of immunogenic molecules. FIGS. 6A and 6B are graphs of the amount of ATP released in the culture media from cells treated with 20 μM or 100 μM CKS1 for 6 hours. Means±SEM, N=3, Welch's t-test (*p<0.05, ***p<0.005). FIGS. 6C and 6D are graphs of the amount of HMGB-1 released in the culture media from cells were treated with 100 μM CKS1 for 6 hours. Means±SEM, N=3, Welch's t-test (*p<0.05).

FIGS. 7A-7C show that CKS1 induces necrosis and suppresses tumor growth in vivo. CKS1 was injected intratumorally in established 4T1 tumors. After 24 hours, tumors were excised and were stained (FIG. 7A). The white area indicates necrosis caused by CKS1. Representative of N=3. FIGS. 7B and 7C are graphs of tumor volume of 4T1 tumors and CT26 tumors, respectively. 40 mg/kg CKS1 was applied daily starting at day 7. Means±SEM, 4T1 control N=15, 4T1 CKS1 treatment N=15, CT26 control N=10, CT26 CKS1 treatment N=10. Welch's t-test was performed for each time point (*p<0.05, ***p<0.005).

FIG. 8 is a chart of LDH release from 4T1 cells treated with peptides. LDH is an indicator of necrotic cell death; higher values indicate increased cancer cell death. For the corresponding sequences, please refer to Table 1.

FIGS. 9A-9C are graphs of cytotoxic activity as measured by LDH release for the indicated peptides. The dots represent the mean±SEM (N=3). The lines represent the dose-response curve fitted to the data using four parameter logistic regression. NF10—SEQ ID NO: 9; NF27—SEQ ID NO: 26; NF28—SEQ ID NO: 27; NF29—SEQ ID NO: 28; NF30—SEQ ID NO: 29; NF31—SEQ ID NO: 30; NF32—SEQ ID NO: 31; NF33—SEQ ID NO: 32; NF34—SEQ ID NO: 33; NF35—SEQ ID NO: 34; NF36—SEQ ID NO: 35; NF37—SEQ ID NO: 36; NF38—SEQ ID NO: 37; NF39—SEQ ID NO: 38; NF40—SEQ ID NO: 39; NF41—SEQ ID NO: 40.

FIGS. 10A-10C show the cytotoxic activity of NF27 in cancer cells. FIG. 10A is graphs of the cell viability of different cancer cell lines treated with 5 different concentrations of NF27 for 48 hours. FIG. 10B is a chart summarizing the GI₅₀ (the concentration required to inhibit 50% of cell growth) of each cell line. FIG. 10C is a table of the EC50 determined by LDH assay for cancer cells types not included in the screens characterized in FIGS. 10A and 10B.

FIGS. 11A-11C show the effects of NF27 on established tumors. FIG. 11A shows the effects of NF27 on CT26 murine colorectal tumors. FIGS. 11B and 11C show the effects of NF27 on HT29 human colorectal tumors established in immunodeficient mice.

### DETAILED DESCRIPTION

Oncolytic peptides induce cancer cell death and generally have no significant toxicity to non-cancerous cells due to differences in cell membrane architecture. Healthy cells maintain an asymmetric distribution of phospholipids in the cell membrane; the outer membrane is mostly composed of neutral phospholipids, while negatively charged phospholipids are sequestered in the inner membrane. In cancer cells, the machinery to maintain the asymmetric distribution is frequently disrupted. Therefore, the cell surface of cancer cells often has higher negative charge compared to that of healthy cells.

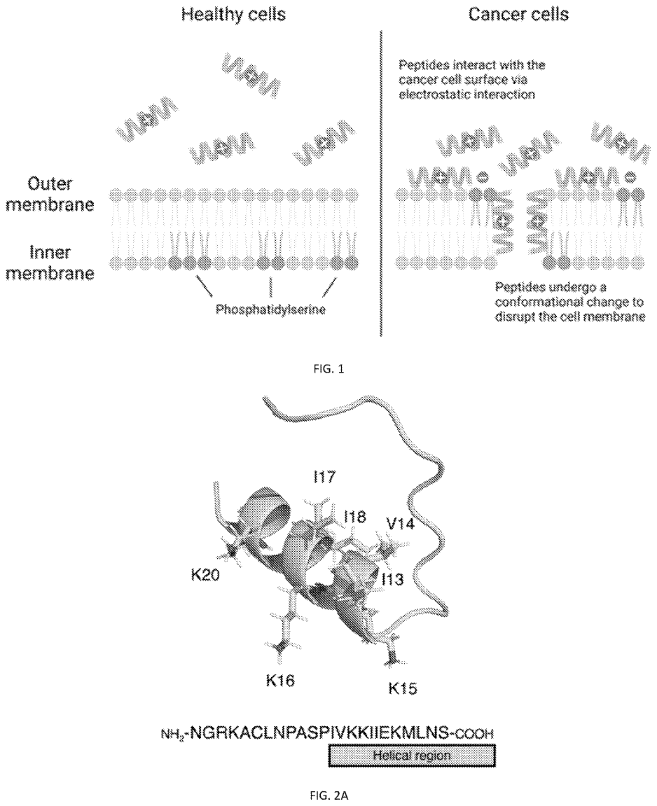

Oncolytic peptides typically have an amphipathic α-helix and a net positive charge. The positively charged regions of the peptides interact with the negatively charged molecules on the cancer cell membrane, such as phosphatidylserine (PS) and glycosylated proteins, via electrostatic interactions (FIG. 1). The peptides are thought to undergo conformational changes, for example when the concentration on the surface exceeds a certain threshold, to disrupt the membrane and eventually induce cell death. Three major models have been proposed to explain the cell penetration step. In the barrel pore model, the peptides span through the membrane to form a pore. The toroidal pore model proposes that the peptides interact with the lipids in the cell membrane to form a pore composed of peptides and lipids. In the carpet model, the peptides cover the surface of the cell membrane, and carve out the lipids by forming micelle-like particles.

The present disclosure provides oncolytic peptides designed with different characteristics in terms of charge, hydrophobicity, and the length of the α-helical region from that of CKS1 (CKS1) which, as described herein, was found to induce cancer cell death and suppress tumor growth. The disclosed oncolytic peptides have much higher potency than CKS1, even at concentrations as low as 5 μM, as demonstrated in by an LDH release assay.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

A “chemotherapeutic agent,” as used herein, refers to a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy. Examples of chemotherapeutic agents include, but are not limited to: cyclophosphamide, methotrexate, 5-fluorouracil, doxorubicin, docetaxel, daunorubicin, bleomycin, vinblastine, dacarbazine, cisplatin, carboplatin, paclitaxel, nab-paclitaxel, raloxifene hydrochloride, tamoxifen citrate, abemacicilib, everolimus, alpelisib, anastrozole, pamidronate, anastrozole, exemestane, capecitabine, epirubicin hydrochloride, eribulin mesylate, erlotinib, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, trastuzumab, temozolomide, rapamycin, and tucatinib.

The term “contacting” as used herein refers to bring or put in contact, to be in or come into contact. The term “contact” as used herein refers to a state or condition of touching or of immediate or local proximity. Contacting to a target destination, such as, but not limited to, an organ, tissue, cell, or tumor, may occur by any means of administration known to the skilled artisan.

A “peptide” or “polypeptide” is a linked sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide,” “oligopeptide,” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state.

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg). Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine. Aromatic amino acids may be sub-divided into two sub-groups: the “nitrogen ring sub-group” consisting of histidine and tryptophan and the “phenyl sub-group” consisting of phenylalanine and tyrosine.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is retained by the peptide.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), BhPro (β-homoproline), BhPhe (β-homophenylalanine) and Bip (β,β diphenylalanine), and Ida (Iminodiacetic acid).

The term “pharmaceutically acceptable salt” in the context of the present invention (pharmaceutically acceptable salt of a peptide described herein) refers to a salt which is not harmful to a patient or subject to which the salt in question is administered. It may suitably be a salt chosen, e.g., among acid addition salts and basic salts. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the peptides may also be quaternized with alkyl chlorides, bromides, and iodides such as methyl, ethyl, propyl, isopropyl, butyl, lauryl, myristyl, stearyl and the like. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3^{rd} edition, James Swarbrick (Ed.), Informa Healthcare USA (Inc.), NY, USA, 2007, and in J. Pharm. Sci. 66:2 (1977).

As used herein, the terms “providing,” “administering,” and “introducing,” are used interchangeably herein and refer to the placement of the peptides or compositions of the disclosure into a subject by a method or route which results in at least partial localization to a desired site. The peptides or compositions can be administered by any appropriate route which results in delivery to a desired location in the subject.

The term “solvate” in the context of the present invention refers to a complex of defined stoichiometry formed between a solute (the peptide or pharmaceutically acceptable salt thereof described) and a solvent. The solvent in this connection may, for example, be water, ethanol, or another pharmaceutically acceptable, typically small-molecular organic species, such as, but not limited to, acetic acid or lactic acid. When the solvent in question is water, such a solvate is normally referred to as a hydrate.

A “subject” or “patient” may be human or non-human and may include, for example, animal strains or species used as “model systems” for research purposes, such a mouse model. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, “treat,” “treating” and the like means a slowing, stopping, or reversing of progression of a disease or disorder when provided a peptide or composition described herein to an appropriate subject. The term also includes a reversing of the progression of such a disease or disorder to a point of eliminating or greatly reducing the disease. As such, “treating” means an application or administration of the peptides or compositions described herein to a subject, where the subject has a disease or a symptom of a disease, where the purpose is to cure, heal, alleviate, relieve, alter, remedy, ameliorate, improve, or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. PEPTIDES

The present disclosure provides peptides, or a pharmaceutically acceptable salt or solvate thereof, comprising an amino acid sequence have one or more amino acid substitutions, additions, or deletions compared to NGRKACLNPASPIVKKIIEKMLNS (SEQ ID NO: 2). The one or more amino acid substitutions, additions or deletions may confer desirable structural and functional characteristics not in SEQ ID NO: 2. For example, the substitutions, additions, or deletions may increase the stability or improve the alpha-helical nature of the peptides or increase the overall oncolytic nature of the peptides (e.g., increase membrane disruption, increase cancer cell death, and the like).

An amino acid “substitution” refers to the substitution or replacement of one amino acid at a given position or residue by another amino acid at the same position or residue within a polypeptide sequence. The amino acid substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure. Examples of conservative amino acid substitutions include substitutions of amino acids within the sub-groups described elsewhere, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into or deleted from amino acid sequence SEQ ID NO: 2. Any number of suitable amino acids can be inserted into or deleted from the amino acid sequence. In this respect, at least one amino acid (e.g., 2 or more, 5 or more, or 10 or more amino acids), but not more than 20 amino acids (e.g., 18 or less, 15 or less, or 12 or less amino acids), can be inserted into or deleted from the amino acid sequence. For example, 1-10 amino acids (e.g., 1, 2, 3, 4, 5, 6, 7, 8, 9, or 10 amino acids) may be inserted into or deleted from the amino acid sequence. In this respect, the amino acid(s) can be inserted into or deleted from any suitable location.

In some embodiments, the peptide comprises an amino acid sequence of NGRKAX₁X₂NPAX₃PX₄X₁₃X₅X₆X₁₄X₁₅X₇X₈X₉X₁₀X₁₁X₁₂ (SEQ ID NO: 41), wherein: X₁ is K, A, L, N or C; X₂ is K or L; X₃ is S, K, or R; X₄ is I, R, K or F; X₅, X₆, X₈ are each independently K or R; X₇ is K, R, or E; X₉ is M, I, or F; X₁₀ is L, R, K, or F; X₁₁ is N, R, or K; X₁₂ is S or I; X₁₃ is V or F; and X₁₄ and X₁₅ are each independently I or F.

In some embodiments, X₄ and X₁₃ are F. In some embodiments, X₄ is I and X₁₃ is V.

In some embodiments, X₉ and X₁₀ are F. In some embodiments, X₄, X₉, X₁₀, and X₁₃ are F. In some embodiments, X₉ and X₁₀ are F, X₄ is I, and X₁₃ is V.

In some embodiments, X₉ is I and X₁₀ is L. In some embodiments, X₉ is I, X₁₀ is L, X₄ is I, and X₁₃ is V. In some embodiments, X₉ is I, X₁₀ is L, and X₄ and X₁₃ are F.

In some embodiments, X₁₄ and X₁₅ are I. In some embodiments, X₁₄ and X₁₅ are I and X₄ and X₁₃ are F. In some embodiments, X₄, X₁₄, and X₁₅ are I and X₁₃ is V.

In some embodiments, X₁₄ and X₁₅ are F. In some embodiments, X₄, X₁₃, X₁₄, and X₁₅ are F. In some embodiments, X₁₄ and X₁₅ are F, X₄ is I, and X₁₃ is V.

In some embodiments, X₅, X₆, X₇, and X₈ are K. In some embodiments, X₁₁ is K and X₁₂ is S. In some embodiments, X₁ is K and X₂ is L. In some embodiments, X₃ is S.

In some embodiments, the proline at position 12 is absent.

In some embodiments, the peptide comprises an amino acid sequence of NGRKAX₁X₂NPAX₃PX₄VX₅X₆IIX₇X₈X₉X₁₀X₁₁X₁₂ (SEQ ID NO: 1), wherein: X₁ is K, A, L, N or C; X₂ is K or L; X₃ is S, K, or R; X₄ is I, R or K; X₅, X₆, X₈ are each independently K or R; X₇ is K, R, or E; X₉ is M or I; X₁₀ is L, R, or K; X₁₁ is N, R, or K; and X₁₂ is S or I.

In some embodiments, X₁ is C. In some embodiments, X₂ is L. In some embodiments, X₁ is C and X₂ is L.

In some embodiments, X₁ is K. In some embodiments, X₂ is K. In some embodiments, X₁ and X₂ are K.

In some embodiments, X₃ is S. In some embodiments, X₃ is S, X₁ is C, and X₂ is L. In some embodiments, X₃ is S and X₁ and X₂ are K.

In some embodiments, X₉ is I. In some embodiments, X₉ is I and X₃ is S. In some embodiments, X₉ is I, X₃ is S, X₁ is C, and X₂ is L. In some embodiments, X₉ is I, X₃ is S, and X₁ and X₂ are K.

In some embodiments, X₁₂ is I. In some embodiments, X₁₁ is K and X₁₂ is I. In some embodiments, X₁₀ is K. In some embodiments, X₁₀, X₁₁, and X₁₂ are K.

In some embodiments, X₅, X₆, and X₈ are R. In some embodiments, X₇ and X₁₁ are R. In some embodiments, X₅, X₆, X₇, X₈, and X₁₁ are R. In some embodiments, X₉ and X₁₂ are I. In some embodiments, X₅, X₆, and X₈ are R and X₉ and X₁₂ are I. In some embodiments, X₇ and X₁₁ are R and X₉ and X₁₂ are I. In some embodiments, X₁₀ is R. In some embodiments, X₅, X₆, X₇, X₈, X₁₀, X₁₁ are R. In some embodiments, X₃ and X₄ are R.

In some embodiments, X₅, X₆, and X₈ are K. In some embodiments, X₇ is K. In some embodiments, X₅, X₆, X₇, and X₈ are K. In some embodiments, X₉ is I and X₁₀ is L. In some embodiments, X₅, X₆, X₇, and X₈ are K, X₉ is I, and X₁₀ is L. In some embodiments, X₁₁ is K and X₁₂ is I. In some embodiments, X₅, X₆, X₇, and X₈ are K, X₁₁ is K, and X₁₂ is I. In some embodiments, X₅, X₆, X₇, X₈, X₁₀, and X₁₁ are K. In some embodiments, X₅, X₆, X₇, X₈, and X₁₁ are K and X₁₀ is L.

In some embodiments, the proline at position 9 is absent. In some embodiments, the proline at position 12 is absent. In some embodiments, the proline at position 9 and the proline at position 12 are absent.

In some embodiments, X₅, X₆, X₇, and X₈ are K. In some embodiments, either the proline at position 9 or the proline at position 12 is absent and X₅, X₆, X₇, and X₈ are K. In some embodiments, X₉ is I. In some embodiments, X₁ is C. In some embodiments, X₁ is any amino acid except C. In some embodiments, X₁ is K, A, L, or N. In some embodiments, X₂ is L. In some embodiments, X₁ is C and X₂ is L. In some embodiments, X₁ is K and X₂ is L. In some embodiments, X₁ is A and X₂ is L. In some embodiments, X₁ and X₂ are L. In some embodiments, X₁ is N and X₂ is L. In some embodiments, X₁ and X₂ are K.

In some embodiments, X₃ is S and X₄ is I. In some embodiments, X₁ is K, A, L, or N, X₂ is L, X₃ is S, X₄ is I, and X₅, X₆, X₇, and X₈ are K. In some embodiments, X₁ is K, A, L, or N, X₂ is L, X₃ is S and X₄ is I. In some embodiments, X₁ is K, A, L, or N, X₂ is L, X₃ is S, X₄ is I, and X₅, X₆, X₇, and X₈ are K.

In some embodiments, X₁₀ is L, X₁₁ is N or K, and X₁₂ is S or I. In some embodiments, X₁₀ is L, X₁₁ is K, and X₁₂ is S. In some embodiments, X₁₀ is L, X₁₁ is N, and X₁₂ is S. In some embodiments, X₁₀ is L, X₁₁ is K, and X₁₂ is I. In some embodiments, X₁₀ is K, X₁₁ is K, and X₁₂ is I.

In some embodiments, X₃ and X₄ are K. In some embodiments, X₁₀ and X₁₁ are K and X₁₂ is I.

In some embodiments, the prolines at positions 9 and 12 are absent and X₁ is C and X₂ is L. In some embodiments, X₃ is S or A and X₄ is K or I. In some embodiments, X₁₀ and X₁₁ are K, and X₁₂ is I. In some embodiments, X₁₀ is L, X₁₁ is N, and X₁₂ is S.

In some embodiments, the peptide has one or more amino acid substitutions, additions, or deletions compared to NGRKACLNPASIVKKIIKKILKS (SEQ ID NO: 9). In some embodiments, the one or more amino acid substitutions, additions, or deletions comprise a substitution for the cysteine at position 6.

In some embodiments, the peptides comprises, consists of, or consists essentially of one or more of the following: NGRKACLNPASPIVKKIIKKMLNS (SEQ ID NO: 3); NGRKACLNPASPIVKKIIKKILNS (SEQ ID NO: 4); NGRKACLNPASPIVKKIIKKILKS (SEQ ID NO: 5); NGRKACLNPASPIVKKIIKKILKI (SEQ ID NO: 6); NGRKACLNPASPKVKKIIKKIKKI (SEQ ID NO: 7); NGRKACLNPASIVKKIIKKILNS (SEQ ID NO: 8); NGRKACLNPASIVKKIIKKILKS (SEQ ID NO: 9); NGRKACLNPASIVKKIIKKILKI (SEQ ID NO: 10); NGRKACLNPASKVKKIIKKIKKI (SEQ ID NO: 11); NGRKACLNPAKKVKKIIKKIKKI (SEQ ID NO: 12); NGRKAKKNPASPIVKKIIEKMLNS (SEQ ID NO: 13); NGRKAKKNPASPIVKKIIKKILKI (SEQ ID NO: 14); NGRKAKKNPASPKVKKIIKKIKKI (SEQ ID NO: 15); NGRKAKKNPASIVKKIIKKILKI (SEQ ID NO: 16); NGRKAKKNPASKVKKIIKKIKKI (SEQ ID NO: 17); NGRKAKKNPAKKVKKIIKKIKKI (SEQ ID NO: 18); NGRKACLNASIVKKIIKKILNS (SEQ ID NO: 19); NGRKACLNAKKVKKIIKKIKKI (SEQ ID NO: 20); NGRKAKKNAKKVKKIIKKIKKI (SEQ ID NO: 21); NGRKACLNPASPIVRRIIERMLNS (SEQ ID NO: 22); NGRKACLNPASPIVRRIIRRILRI (SEQ ID NO: 23); NGRKACLNPARRVRRIIRRIRRI (SEQ ID NO: 24); NGRKAKKNPARRVRRIIRRIRRI (SEQ ID NO: 25); NGRKAKLNPASIVKKIIKKILKS (SEQ ID NO: 26); NGRKAALNPASIVKKIIKKILKS (SEQ ID NO: 27); NGRKALLNPASIVKKIIKKILKS (SEQ ID NO: 28); NGRKANLNPASIVKKIIKKILKS (SEQ ID NO: 29); NGRKAKLNPASFFKKIIKKILKS (SEQ ID NO: 30); NGRKAKLNPASIVKKFFKKILKS (SEQ ID NO: 31); NGRKAKLNPASIVKKIIKKFFKS (SEQ ID NO: 32); NGRKAKLNPASFFKKFFKKILKS (SEQ ID NO: 33); NGRKAKLNPASIVKKFFKKFFKS (SEQ ID NO: 34); NGRKAKLNPASFFKKIIKKFFKS (SEQ ID NO: 35); NGRKAKLNPASFFKKFFKKFFKS (SEQ ID NO: 36).)

In some embodiments, the peptide comprises an amino acid sequence of ASIVKKIIKKILKS (SEQ ID NO: 37). In some embodiments, the peptide consists of an amino acid sequence of ASIVKKIIKKILKS (SEQ ID NO: 37). In some embodiments, the peptide comprises an amino acid sequence of ASIVKKIIKKILKS (SEQ ID NO: 37) and is 50 total amino acids or less (e.g., 45, 40, 35, 30, 25, 20 or less). In some embodiments, the peptide comprises an amino acid sequence of ASIVKKIIKKILKS (SEQ ID NO: 37) and is 25 total amino acids or less. In some embodiments, the peptide is 15 total amino acids, 16 total amino acids, 17 total amino acids, 18 total amino acids, 19 total amino acids, 20 total amino acids, 21 total amino acids, 22 total amino acids, 23 total amino acids, 24 total amino acids, or 25 total amino acids.

In some embodiments, the peptide comprises, consists of, or consists essentially of one or more of the following amino acid sequences: NGRKACLNPASIVKKIIKKILKS (SEQ ID NO: 9); NGRKAKLNPASIVKKIIKKILKS (SEQ ID NO: 26); NGRKAALNPASIVKKIIKKILKS (SEQ ID NO: 27); NGRKALLNPASIVKKIIKKILKS (SEQ ID NO: 28); NGRKANLNPASIVKKIIKKILKS (SEQ ID NO: 29); ASIVKKIIKKILKS (SEQ ID NO: 37); RKAKLNPASIVKKIIKKILKS (SEQ ID NO: 38); AKLNPASIVKKIIKKILKS (SEQ ID NO: 39); and LNPASIVKKIIKKILKS (SEQ ID NO: 40).

In some embodiments, the peptides are modified to stabilize them, to facilitate their uptake and/or absorption, or to improve any other characteristic or property of the peptides (e.g., solubility) that is known to one of skill in art. In some embodiments, the peptide is structurally stabilized or modified to maintain its native secondary structure. For example, the peptides may be stabilized to maintain or improve the alpha-helical nature of the peptides. Stabilized peptides include stapled or stitched peptides in which using any number of reaction conditions and/or catalysts to facilitate a reaction of side chains present in a peptide chain, e.g., crosslinking or ring-closing reactions. In some embodiments, the peptides are modified or configured to facilitate replacement of reactive/unstable amino acids, notably cysteines, to improve chemical stability

The peptides may also be modified by the addition of: radioactive atoms; detectable labels (e.g., radioactive labels, dyes, fluorescent moieties, chemiluminescent moieties, quantum dots); affinity tags (e.g., His tag, biotin); PEG moieties; carbohydrates (e.g., glycosylation, hesylation); and organic molecules (e.g., alkylation, acetylation, acylation).

The present disclosure also provides for nonpeptide compounds that mimic peptide sequences (“mimetics”), synthesis of which are known in the art. Peptide mimetics that are structurally related to therapeutically useful peptides may be used to produce an equivalent or enhanced therapeutic or prophylactic effect. Generally, peptidomimetics are structurally similar to the peptide of interest, but have one or more peptide linkages optionally replaced by linkages such as —CH₂NH—, —CH₂S—, —CH₂CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and the like.

The peptides can be produced by chemical synthesis methods well known in the art, including, for example, solid phase peptide synthesis (SPPS). See, for example, Fields et al., Chapter 3 in Synthetic Peptides: A User's Guide, ed. Grant, W. H. Freeman & Co., New York, N.Y., 1992, p. 77. Alternatively or additionally, the peptides can be synthesized by well-known recombinant DNA techniques.

Embodiments of the present disclosure also include a polynucleotide encoding any of the peptides of the present disclosure. In accordance with these embodiments, the present disclosure includes an expression vector comprising any of the polynucleotides encoding a peptide of the present disclosure. In some embodiments, the expression vector is suitable for manufacturing a peptide disclosed herein or for delivery of the peptide to a subject, tissue, or cell.

In certain embodiments, the nucleic acid sequence is in the form of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 4th edition, Cold Spring Harbor Press, Cold Spring Harbor, N.Y. (2012), and Ausubel et al., Current Protocols in Molecular Biology, Greene Publishing Associates and John Wiley & Sons, New York, N.Y. (1994)).

In addition to the nucleic acid encoding a peptide as disclosed here, the vector desirably comprises expression control sequences, such as promoters, enhancers, polyadenylation signals, transcription terminators, internal ribosome entry sites (IRES), and the like, that provide for the expression of the antibody-encoding nucleic sequence in a host cell. Exemplary expression control sequences are known in the art and described in, for example, Goeddel, Gene Expression Technology: Methods in Enzymology, Vol. 185, Academic Press, San Diego, Calif. (1990).

A vector comprising a nucleic acid sequence encoding a peptide disclosed herein may be introduced into a host cell that is capable of expressing the polypeptides encoded thereby, including any suitable prokaryotic or eukaryotic cell. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5a, DH10, MC1061 (ATCC No. 53338), and CC102). Suitable eukaryotic cells are known in the art and include, for example, yeast cells, insect cells, and mammalian cells. Examples of suitable yeast cells include those from the genera Hansenula, Kluyveromyces, Pichia, Rhinosporidium, Saccharomyces, and Schizosaccharomyces. Suitable insect cells include Sf-9 and HIS cells (Invitrogen, Carlsbad, Calif.) and are described in, for example, Kitts et al., Biotechniques, 14:810-817 (1993); Lucklow, Curr. Opin. Biotechnol., 4:564-572 (1993); and Lucklow et al., J. Virol., 67:4566-4579 (1993). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of such cells are well known in the art (see, e.g., Ausubel et al., eds., Short Protocols in Molecular Biology, 5th ed., John Wiley & Sons, Inc., Hoboken, N.J. (2002)). Preferably, the mammalian cell is a human cell.

In accordance with the above embodiments, the present disclosure provides peptides comprising desirable oncolytic functional characteristics, including inhibiting angiogenesis, rupturing cell membranes, inducing apoptotic pathways, inducing cancer cell death, decreasing tumor growth, and/or stimulating anti-cancer immunity. In some embodiments, the peptides may also confer desirable solubility characteristics. For example, the disclosed peptides are soluble in aqueous solutions in concentrations in excess of 30 mg/ml, well above the approximate 100 μM (0.27 mg/ml) concentration required for in vitro efficacy. Given that other reported oncolytic peptides, such as LTX-315, are not soluble in water, the peptides provide increased ease of use in formulations, compositions, and therapeutics development.

### 3. COMPOSITIONS

Disclosed herein are compositions comprising the peptides, a polynucleotide encoding any of the peptides, as described above. The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any appropriate manner of administration, and thus administered, including for example, oral, nasal, intravenous, intraarterial, intravaginal, epicutaneous, sublingual, intracranial, intradermal, intraperitoneal, subcutaneous, intramuscular, intratumoral, administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage. The route or administration and the form of the composition usually dictates the type of carrier to be used.

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

### 4. METHODS

The disclosed peptides and compositions may be used in various methods, including methods for treating or preventing a disease or disorder in a subject, methods for lysing cancer cell membranes, stimulating the anti-tumoral immunity, and/or methods for inducing cancer cell death.

In one embodiment, provided is a method for treating or preventing a disease or disorder in a subject comprising administering an effective amount of the peptides or compositions described herein to the subject. In some embodiments, the subject is a human. In some embodiments, the methods comprise providing or administering to the subject a nucleic acid encoding the peptides.

The peptides of the present disclosure disrupt cell membranes, particularly cell membranes of cancer cells, resulting in cell death. Thus, the disclosed peptides are useful in the treatment of diseases and disorders in which diseased cells have an altered cell membrane composition, for example a higher surface negative charge compared to that of healthy cells, generally cells which have a proliferative disease or disorder.

In some embodiments, the disease or disorder is cancer. In some embodiments, the cancer comprises a solid tumor. In some embodiments, the cancer is metastatic cancer. In some embodiments, the disclosed peptides, compositions, or methods result in decreased tumor growth. In some embodiments, the disclosed peptides, compositions, or methods prevent tumor recurrence. In some embodiments, the disclosed peptides, compositions, or methods inhibit proliferation of a cancer cell. In some embodiments, the disclosed peptides, compositions, or methods induce cancer cell death and related pathways (e.g., apoptotic pathways). In some embodiments, the disclosed peptides induce cancer cell death with no significant toxicity to non-cancerous cells. In some embodiments, the disclosed peptides, compositions, or methods stimulate anti-cancer immunity, e.g., through the release of immunogenic molecules (e.g., neoantigens, ATP, and high mobility group box 1 (HMGB1)). In some embodiments, the disclosed peptides inhibit angiogenesis and/or rupture cancer cell membranes.

The methods disclosed herein may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. In some embodiments, the cancer is transdermally accessible. For example, those cancers with transdermally accessible tumors, including but not limited to, skin cancer (e.g., basal cell carcinoma, squamous cell carcinoma and melanoma), colorectal cancer (e.g., rectal cancer, metastatic colorectal cancer) and breast cancer (e.g., triple negative breast cancer).

Peptides of the present disclosure may be administered to a subject by a variety of methods. In any of the uses or methods described herein, administration may be by various routes known to those skilled in the art, including without limitation oral, inhalation, intravenous, intraarterial, intramuscular, subcutaneous, systemic, and/or intraperitoneal administration to a subject in need thereof. In some embodiments, the peptides or compositions as disclosed herein may be administered by parenteral administration (including, but not limited to, subcutaneous, intramuscular, intravenous, intraarterial (e.g., hepatic artery), intraperitoneal, intracardiac, and intraarticular injections). In some embodiments, the peptides or compositions as disclosed herein are administered intratumorally or adjacent to the site of the tumor.

The terms “effective amount” or “therapeutically effective amount,” as used herein, refer to a sufficient amount of the peptides or compositions disclosed herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the disclosed peptides or compositions required to provide a clinically significant decrease in cancer and cancer related symptoms.

The amount of the peptides or compositions of the present disclosure required for use in treatment or prevention will vary not only with the particular peptide or composition selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of a peptide of the present invention, or composition thereof, can be determined by comparing their in vitro activity, and in vivo activity in animal models.

Dosage amount and interval may be adjusted individually to provide plasma levels of the active agent which are sufficient to maintain the modulating effects, or minimal effective concentration (MEC). The MEC will vary for each peptide but can be estimated from in vivo and/or in vitro data. Dosages necessary to achieve the MEC will depend on individual characteristics and route of administration. However, bioassays can be used to determine plasma concentrations. Dosage intervals can also be determined using MEC value. Compositions or peptides should be administered using a regimen, which maintains plasma levels above the MEC for 10-90% of the time, preferably between 30-90% and most preferably between 50-90%. In cases of local administration or selective uptake, the effective local concentration of the peptide may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be also used in veterinary medicine for non-human subjects.

Peptides and compositions disclosed herein can be evaluated for efficacy and toxicity using known methods. For example, the toxicology of a particular peptide may be established by determining in vitro toxicity towards a cell line, such as a mammalian, and preferably human, cell line. The results of such studies are often predictive of toxicity in animals, such as mammals, or more specifically, humans. Alternatively, the toxicity of particular peptides in an animal model, such as mice, rats, rabbits, dogs, or monkeys, may be determined using known methods. The efficacy of a particular peptide may be established using several recognized methods, such as in vitro methods, animal models, or human clinical trials. When selecting a model to determine efficacy, the skilled artisan can be guided by the state of the art to choose an appropriate model, dose, route of administration and/or regime.

A therapeutically effective amount of a peptide disclosed herein, or compositions thereof, may be administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time, wherein one composition includes a peptide of this invention, and the other includes the second agent(s). Alternatively, in other embodiments, the therapy precedes or follows the other agent treatment by intervals ranging from minutes to months.

A wide range of second therapies may be used in conjunction with the peptides of the present disclosure. The second therapy may be a combination of a second therapeutic agent or may be a second therapy not connected to administration of another agent. Such second therapies include, but are not limited to, surgery, immunotherapy (e.g., chimeric antigen receptor (CAR) T-cell or T-cell transfer therapies, cytokine therapy, immunomodulators, cancer vaccines, or administration of antibodies (e.g., monoclonal antibodies), immune receptor agonists, immune checkpoint inhibitors), radiotherapy, oncolytic viral therapies, or chemotherapy. For example, when treating breast cancer, the peptides of the present disclosure may be administered in conjunction with paclitaxel, nab-paclitaxel, and/or doxorubicin.

The second therapy may be administered at the same time as the initial therapy, either in the same composition or in a separate composition administered at substantially the same time as the first composition. In some embodiments, the second therapy may precede or follow the treatment of the first therapy by time intervals ranging from hours to months.

In accordance with the above embodiments, the present disclosure provides peptides comprising desirable characteristics for a cancer therapeutic. The peptides possess a lower risk of drug resistance, fewer side effects, and significant cost saving as compared to many current cancer therapeutics. The disclosed peptides directly attack cancer cell membranes, thereby avoiding many of the sides effects associated with therapeutics which act by modulating a cell surface receptor. Peptides typically have a half-life of a few minutes in human serum. For example, insulin, the most widely used peptide therapeutic, has a half-life of approximately 3 to 10 minutes in the bloodstream. As such, any long-term side effects due to residence time in the subject are also not likely. Finally, the cost of manufacturing the disclosed peptides will likely be significantly less than that of monoclonal antibodies or other biologics because solid phase synthesis can utilize natural amino acids in standard synthesis protocols.

In another embodiment, provided is a method for lysing cancer cell membranes and/or and inducing cancer cell death comprising contacting a cancer cell with an effective amount of the peptides or compositions described herein. The cancer cell may be a cell with any cancer, as described above.

In some embodiments, the cancer cell is in vitro. In some embodiments, the cancer cell is ex vivo. In some embodiments, the cell is in an organism or host, such that introducing the peptides or compositions described herein into the cell comprises administration to a subject. The method may comprise providing or administering to the subject in vivo the peptides or compositions as described elsewhere herein. In some embodiments, the methods comprise providing or administering to the subject a nucleic acid encoding the peptides.

### 5. KITS

In another aspect, the disclosure provides kits comprising at least one disclosed peptide, or a composition comprising the peptide, and instructions for using the peptide or composition.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed peptide and/or product and another agent (e.g., a chemotherapeutic, a monoclonal antibody, a pain reliever, a steroid, an anti-emetic) for delivery to a patient or a cell.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates, flasks).

The kits provided herein are in suitable packaging. Suitable packaging includes, but is not limited to, vials, bottles, jars, flexible packaging, and the like. Individual member components of the kits may be physically packaged together or separately.

### 6. EXAMPLES

### Materials and Methods

Peptides The peptides were synthesized by Genscript (Piscataway, NJ) using a solid-phase peptide synthesis technique. The purity was >90% as verified by HPLC and MS analyses. The peptides were solubilized in water.

Cell culture 4T1 murine mammary carcinoma cells (CRL-2539), CT26 murine colon carcinoma cells (CRL-2638), U2OS human osteosarcoma cells (HTB-96), and NIH/3T3 normal murine fibroblast cells (CRL-1658) were purchased from the American Type Culture Collection (Manassas, VA). MCA205 murine fibrosarcoma cells (SCC173) were purchased from MilliporeSigma (St. Louis, MO). 4T1 cells and MCA205 cells were propagated in RPMI 1640 (Corning, Corning, NY) supplemented with 10% FBS (MilliporeSigma). NIH/3T3 cells and α2OS cells were propagated in DMEM (Thermo Fisher Scientific) supplemented with 10% FBS. HUVECs were propagated in EGM-2 bulletkit (LONZA, Basel, Switzerland). All cell lines were grown in T75 tissue culture flasks under standard culture conditions of 37 °C and 5% CO2 (Sarstedt, Nümbrecht, Germany).

Animal models The protocols used in this study were approved by the Institutional Care and Use Committee at Johns Hopkins Medical Institutions. Four- to eight-week-old female Balb/c mice were obtained from Charles River (Wilmington, MA). For the 4T1 triple breast cancer model, 2.5×10⁴ 4T1 cells were injected in the first mammary fat pad of each mouse. For the CT26 colon carcinoma model, 2.5×10⁴ CT26 cells were injected subcutaneously in the left flank. After 1 week, daily intratumoral treatment was started with 40 mg/kg CKS1. The tumor size was measured by using a caliper, and the volume was calculated by using the formula 0.52×(length)×(width)².

Real-time cell viability assay Three thousand cells were seeded in xCELLigence RTCA E-plate 16 (Agilent, Santa Clara, CA). After the cells adhered to the plate, the media was replaced with serum free media and the cells were treated with CKS1 at the indicated concentration. The viability of the cells was monitored using xCELLigence RTCA analyzer (Agilent).

LDH assay 1.5×10⁴ cells were seeded in a 96 well plate and incubated overnight. The cells were washed once, and the medium was replaced with serum free media. Cells were treated for the indicated time and concentration. CyQUANT LDH cytotoxicity assay (Invitrogen, Carlsbad, CA) was used following the manufacturer's instructions.

BrdU assay A thousand cells were seeded in wells of a 96 well plate. The cells were treated with the indicated concentration immediately after seeding. 48 hours later, 5-bromo-2′-deoxyuridine (BrdU) was added to each well. 24 hours later, BrdU incorporation was quantified using BrdU cell proliferation assay kit (CST, Danvers, MA) following the manufacturer's instructions.

Cell lysis and immunoblotting Cells were lysed in cell lysis buffer (Cell Signaling, #9661) supplemented with protease/phosphatase inhibitor cocktail (Cell Signaling, #5872). The cell extracts were centrifuged and the supernatants were sampled by adding blue loading buffer pack (Cell Signaling, #7722). Samples were boiled at 98 °C for 3 minutes and resolved by SDS-PAGE. Samples were electroblotted on a nitrocellulose membrane using the iBlot gel transfer device (Invitrogen, Waltham, MA) and the iBlot transfer stack (Invitrogen, IB301002). The membrane was blocked with 2% skim milk (LabScientific, Danvers, MA) in TBS-T (Bioland Scientific, Paramount, CA, TBST01-03) and probed with the appropriate primary antibody. After replacing and probing with the appropriate secondary antibodies diluted with skim milk in TBS-T, antibody-antigen complexes were illuminated using ultra digital-ECL substrate solution (Kindle Biosciences, Greenwich, CT) and detected with KwikQuant imager (Kindle Biosciences). Cleaved caspase-3 antibody (Cell Signaling, #9661) and β-tubulin (MilliporeSigma, T8328) were used as primary antibodies. Anti-rabbit IgG, HRP-linked antibody (Cell Signaling, #7074) and Anti-mouse IgG, HRP-linked antibody (Cell Signaling, #7076) were used as secondary antibodies.

Quantification of ATP release 1.5×10⁴ cells of 4T1 cells or CT26 cells in growth media were seeded in wells of a 96 well plate. After 12 hours, the media was replaced with serum free media and the cells were treated with the indicated concentration of peptide for 6 hours and the supernatant was collected from each well. After filtering the supernatant with 0.2 μm pore filter (Corning, 431229), the amount of ATP in the supernatant was quantified using the ENLITEN ATP assay system (Promega, FF2000). Manufacturer's instructions were used with the ENLITEN ATP assay system.

Quantification of HMGB-1 release 1.5×10⁴ cells of 4T1 cells or CT26 cells in growth media were seeded in wells of a 96 well plate. After 12 hours, the media was replaced with serum free media and the cells were treated with the corresponding concentration of peptide. The cells were treated for 6 hours and the supernatant was collected from each well. After filtering the supernatant with 0.2 μm pore filter (Corning, 431229), the amount of HMGB-1 in the supernatant was quantified using the Mouse/Rat HMGB1 ELISA kit (arigo biolaboratories, ARG81310).

H&E staining Tumors were fixed by immersion in 10% neutral buffered formalin (Millipore Sigma) for 48 hours and subsequently dehydrated by immersion in 70%, 90%, and 100% ethanol followed by xylene for two changes of 30 minutes each. Tissues were embedded in paraffin blocks at 58 °C and sectioned at 4 μm using a microtome. Sections were floated in a water bath at 56 °C and embedded onto glass slides and dried overnight. Next, sections were rehydrated by immersion in xylene, followed by 100%, 95%, 70% ethanol, and finally water for two changes of 5 minutes each. For staining, slides were immersed in Gill's Hematoxylin #2 (Millipore Sigma) for 1 minute, immediately rinsed in tap water to prevent overstaining, and immersed in Eosin Y (Millipore Sigma) for 1 minute and rinsed. Finally, slides were again dehydrated, mounted with Citramount, coverslipped, and scanned at 40× with a NanoZoomer slide scanner.

NCI-60 screening The peptides were submitted to the National Cancer Institute (NCI of NIH) Developmental Therapeutics anticancer screening program (DTP) for human tumor cell line assay by NCI-60 screening against a 60 cell panel at five concentration levels (dtp.cancer.gov/discovery_development/nci-60/methodology.htm).

Statistics All statistics analyses were performed using R. Differences between two groups were determined using unpaired two-tailed Welch's t-test and were considered statistically significant when p<0.05. Differences between three or more groups were determined by one-way ANOVA with Tukey-Kramer test. To determine the difference between the control group and the treatment groups, we used one-way ANOVA with Dunnett's test. All experiments were replicated at least three times and the results are expressed as the standard error of the mean.

### Example 1

### CKS1 Structure

CKS1 (SP3019) is a 24-mer peptide (NGRKACLNPASPIVKKIIEKMLNS; SEQ ID NO: 2) derived from a sequence shared in the CSC chemokine family originally discovered as an anti-angiogenic peptide (See, Karagiannis E D, Popel A S. J Cell Biochem. 2008; 104:1356-63).

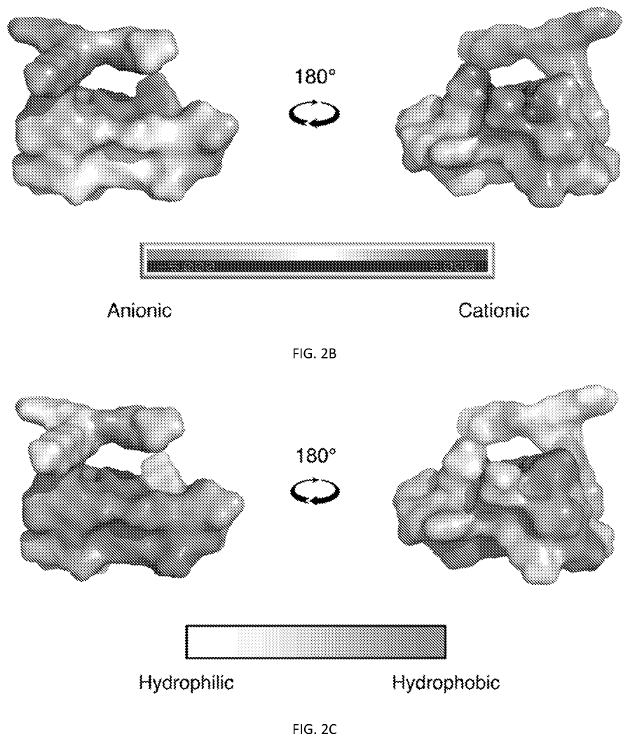

CKS1 was predicted to be an α-helix by a protein structure prediction software AlphaFold 2 (FIG. 2A). The prediction of the protein structure was done using the ColabFold platform, and was visualized by Pymol. CKS1 contains several basic amino acids that make the net charge positive (FIG. 2B). The charged amino acids are concentrated on one side of the helix, and the other side of the helix is formed by hydrophobic amino acids (FIG. 2C).

### Example 2

### CKS1 Function

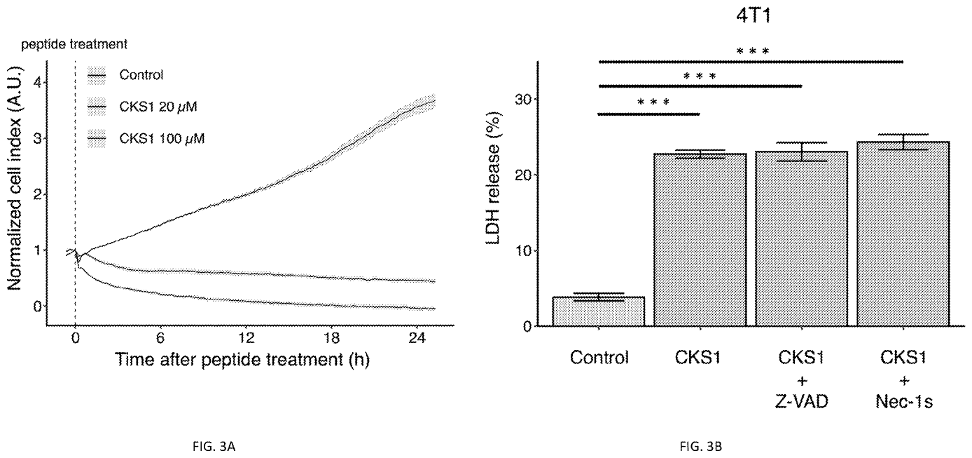

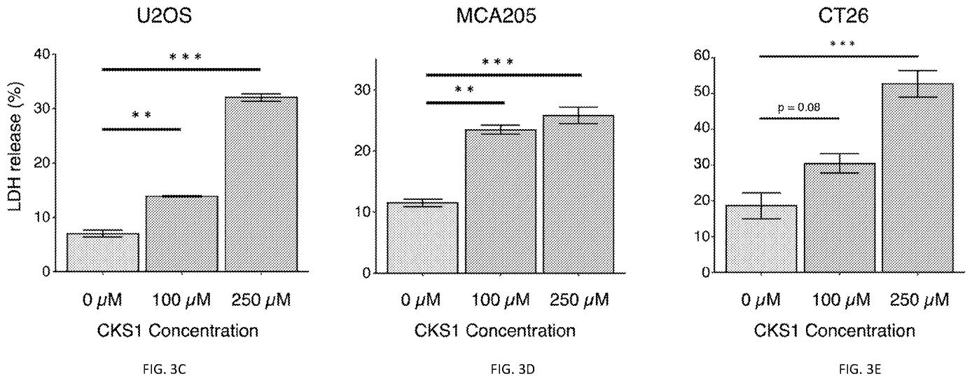

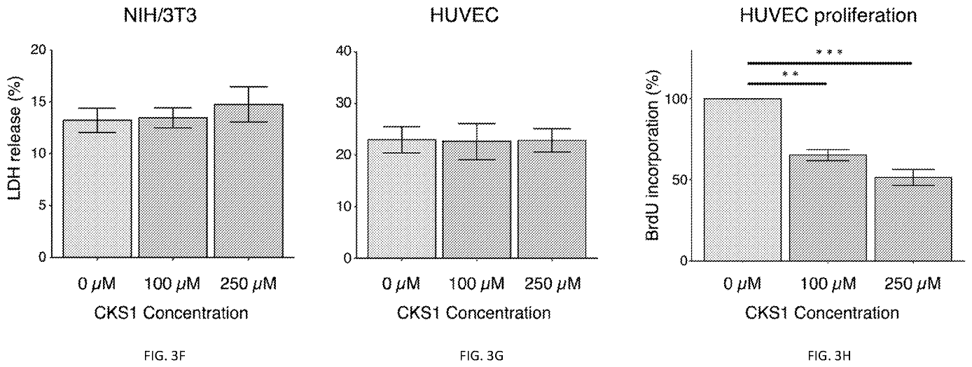

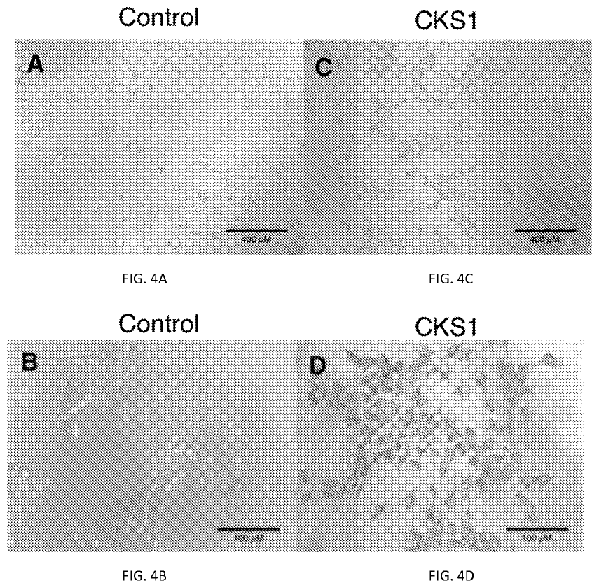

To determine whether CKS1 functions as an oncolytic peptide, the cell viability of 4T1 murine breast cancer cells was observed after CKS1 treatment using xCELLigence Real-Time Cell Analyzer (Agilent). In this method, the binding of cells to gold electrodes is detected as electrical impedance and graphed as arbitrary units of cell index in real time. Cell death, as suggested by a reduction in cell index, was observed within 1 hour of treatment and was dose dependent (FIG. 3A). The release of lactate dehydrogenase (LDH) was also quantified in the culture media after CKS1 treatment. LDH is a cytosolic enzyme which is released outside the cells when the cell membrane is damaged. Cells treated with CKS1 released significant amounts of LDH showing that CKS1 induced damage in the cell membrane of 4T1 cells. Furthermore, pretreatment with the pan-caspase inhibitor Z-VAD or RIPK1 inhibitor necrostatin-1 stable (Nec-1s) did not inhibit the release of LDH, indicating that the release of LDH by CKS1 was independent from these programed cell death pathways (FIG. 3B). The cytotoxicity of CKS1 was not limited to 4T1 cells, and it was found to be effective in several murine and human cancer cell lines: human osteosarcoma U2OS cells, murine fibrosarcoma MCA205 cells, and murine colon carcinoma CT26 cells (FIGS. 3C-3E). Importantly, CKS1 did not induce cell death of NIH/3T3 normal fibroblasts and human umbilical vein endothelial cells (HUVECs) (FIGS. 3F-3G). Although CKS1 did not induce LDH release, CKS1 significantly attenuated cell proliferation of HUVECs as demonstrated by the inhibition of BrdU incorporation (FIG. 3H), indicating that the reported anti-angiogenic property is separate from the cell death-inducing capacity of CKS1.

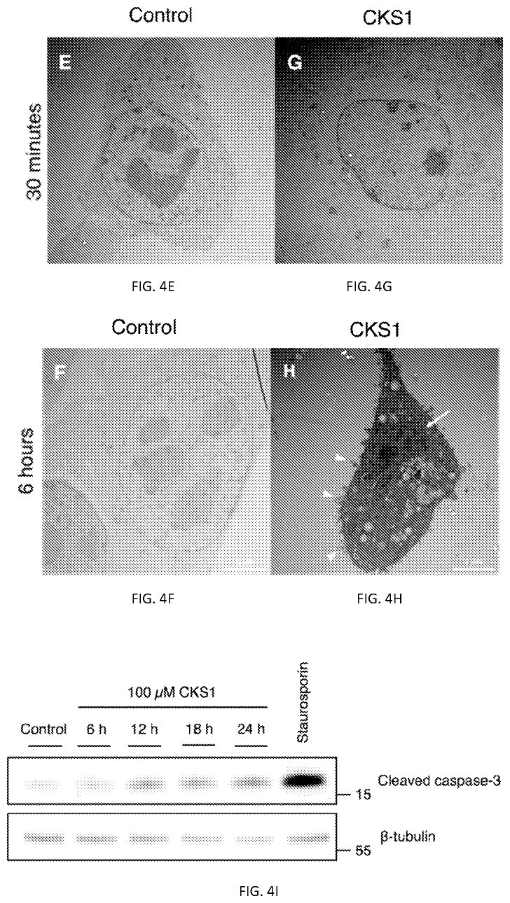

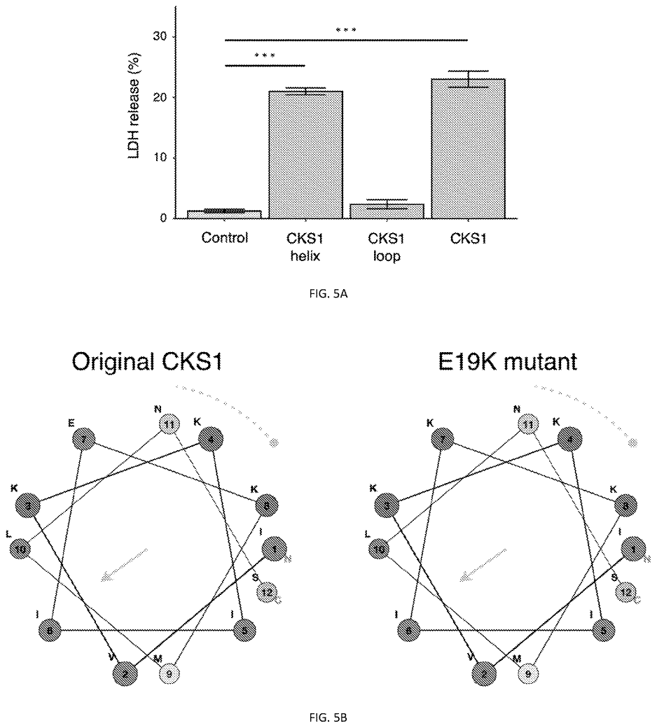

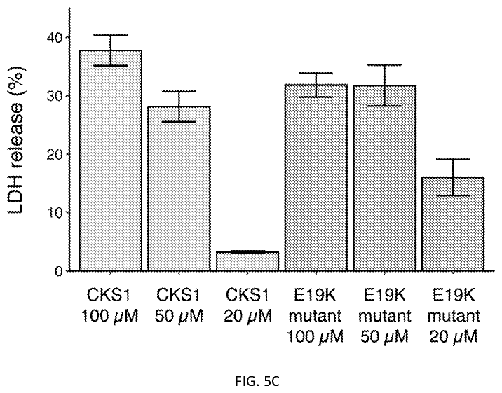

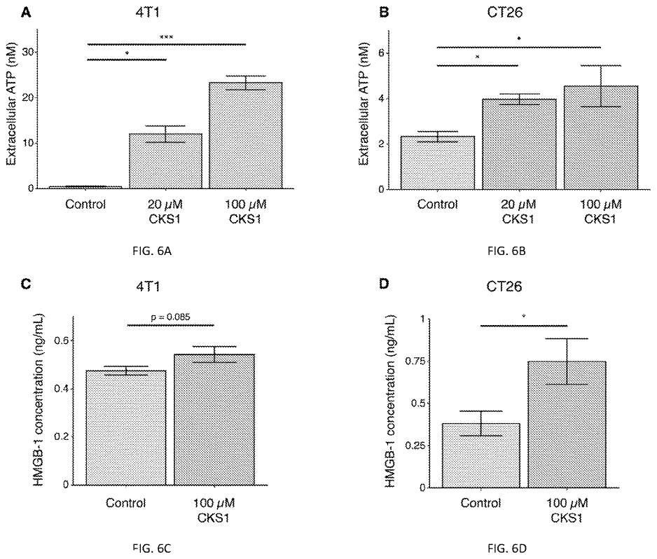

To further identify the mechanism of cell death caused by CKS1, the morphology of the cells after CKS1 treatment was observed. Upon treatment with CKS1, 4T1 cells underwent a significant change in their morphology, characterized by the shrinkage of the cell volume, rough texture of the cell surface, and the release of cellular content (FIGS. 4A-4D). The morphology of the cell membrane was further observed using electron microscopy. The cell membrane of 4T1 cells was disrupted when treated with CKS1 for 30 minutes (FIGS. 4E and 4F). When treated with CKS1 for 6 hours, cells with an apoptotic morphology were observed (FIGS. 4G and 4H). The volume of the cells shrunk when treated with CKS1 for a long period, and apoptotic blebbing was observed. Despite the cell membrane being disrupted, the nuclear envelope of the cells was intact when the cells were treated with CKS1 for 30 minutes. However, when the cells were treated with CKS1 for 6 hours, the nucleus collapsed and the chromatin condensed. It was confirmed that cells underwent apoptosis by quantifying the activity of caspase-3 over the course of 24 hours (FIG. 41). These data indicate that CKS1 induces caspase-independent necrotic cell death by disrupting the cell membrane at early time points, and further induces apoptosis by activating caspase-3 at later time points.

As described above, CKS1 possesses an amphipathic, positively charged α-helix, which is suitable for oncolytic activity. To examine whether the α-helix plays a role in oncolytic activity, two peptides were produced: one that only contains the helical region (IVKKIIEKMLNS; SEQ ID NO: 42) and another that contains the loop region (NGRKACLNPASP; SEQ ID NO: 43). LDH release was measured from 4T1 cells upon treatment with the two peptides. As expected, the helical region of CKS1 induced LDH release from 4T1 cells, while the loop region did not (FIG. 5A). Although the loop region was not involved in the oncolytic activity, the peptide with only the helical region was not soluble in water, while CKS1 shows high solubility in water (data not shown). To further analyze the structure-activity relationship and potentially develop a peptide with higher activity, an E19K mutant peptide was produced. The helical region of CKS1 and the E19K mutant is shown as a helical wheel representation (FIG. 5B). The original CKS1 has a negatively charged glutamic acid on the hydrophilic side. Replacing the glutamic acid with a positively charged lysine will make the overall charge of CKS1 more positively charged with a concentrated positive charge on one side of the helix, which may display higher affinity to the cell surface of cancer cells, resulting in higher oncolytic activity. When treated at a higher concentration, the original peptide and the E19K mutant demonstrated equivalent release of LDH release. However, the E19K mutant showed significantly higher release of LDH at lower concentrations, indicating that the E19K mutant has a higher affinity to the cell surface of cancer cells as expected (FIG. 5C). These data demonstrated that the helical region of CKS1 directly influences the oncolytic activity.

The release of damage associated molecular patterns (DAMPs) such as ATP and HMGB-1 is a representative characteristic of immunogenic cell death. To determine whether cell death caused by CKS1 is immunogenic, the amount of ATP and HMGB-1 released in the culture media upon CKS1 treatment was evaluated. A release of both ATP (FIGS. 6A and 6B) and HMGB-1 (FIGS. 6C and 6D) were observed, indicating that CKS1 induces immunogenic cell death.

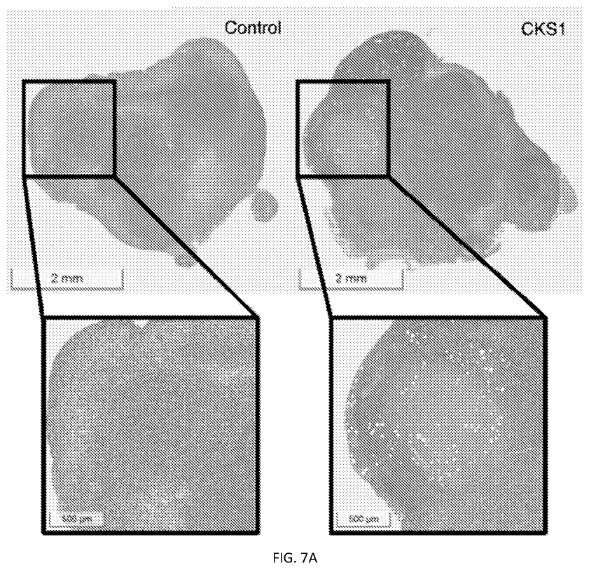

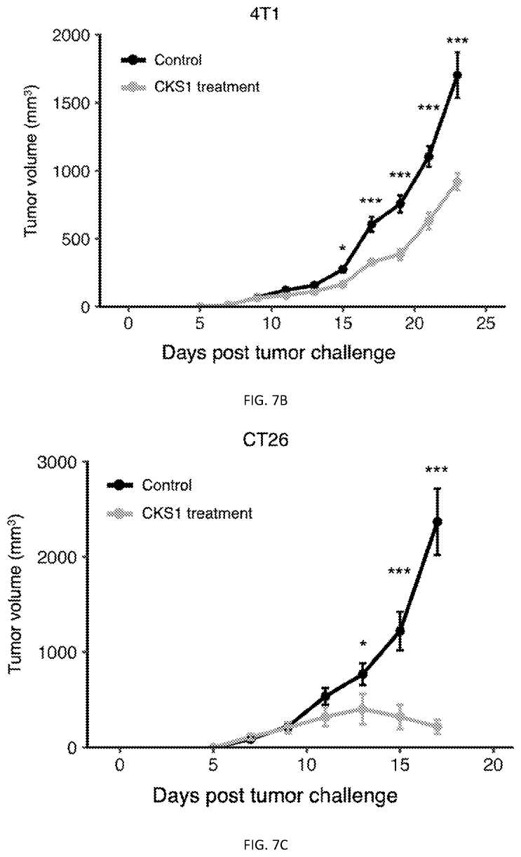

To evaluate whether CKS1 can induce cancer cell death in vivo, CKS1 was intratumorally injected into established 4T1 tumors. Intratumoral injection was chosen to separate the peptide mechanism of action from its transport properties as would be the case with e.g., intraperitoneal or subcutaneous administration. Necrosis was observed in tumors 24 hours after CKS1 treatment (FIG. 7A). Furthermore, growth of both 4T1 tumors and CT26 tumors was inhibited by CKS1 treatment (FIGS. 7B and 7C). Interestingly, CT26 tumors were highly sensitive to CKS1, while 4T1 tumors showed a relatively modest response to CKS1. This may be due to the difference in immunogenicity and the influence on the tumor microenvironment between the two cell lines. 4T1 cells are poorly immunogenic, with lower mutation rates compared to CT26. Accordingly, CT26 cells respond well to immune checkpoint inhibitors, while 4T1 respond poorly.

### Example 3

### Oncolytic Peptides

Since CKS1 was originally not designed as an oncolytic peptide, the sequence was modified to design peptides with better oncolytic activity. As described above, the α-helix is a key component to the functionality of CKS1 and altering the residues in the α-helix may lead to derivative peptides with better properties for cancer treatment. The amino acids selected for editing were those predicted to alter the structure such that the peptide is more favorable as an oncolytic peptide in terms of charge, hydrophobicity, and the length of the α-helix. Table 1 shows the sequences of exemplary peptides.

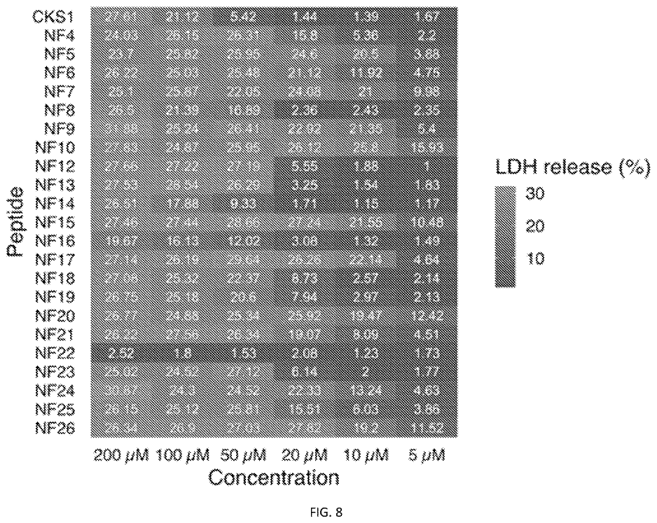

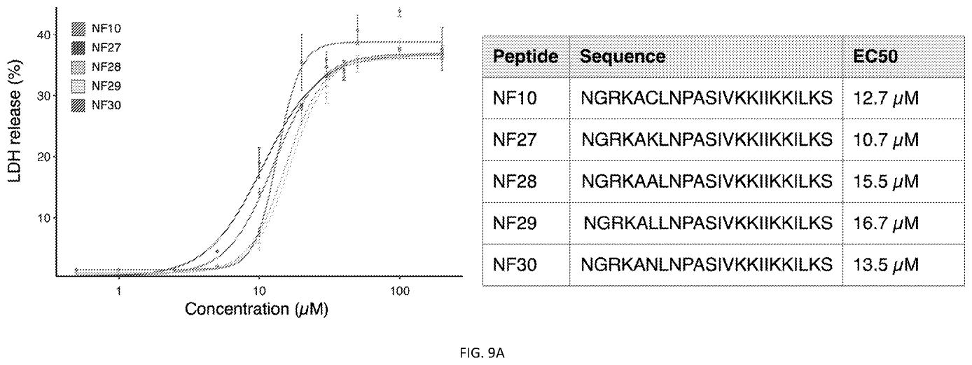

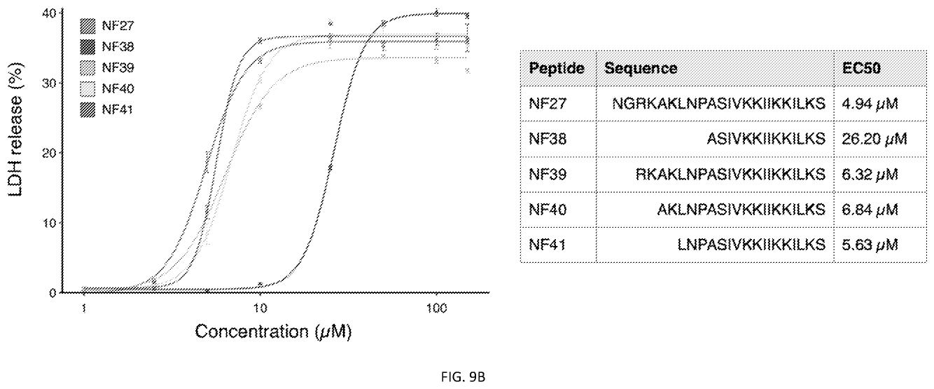

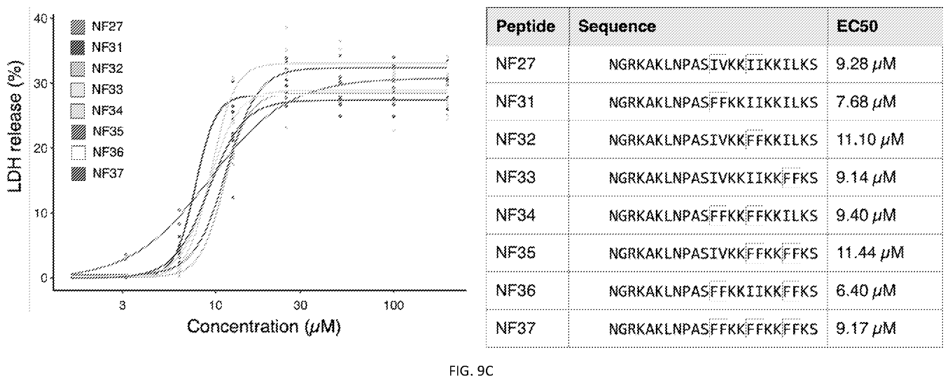

A number of peptides were generated that showed better activity, as measured by LDH release, than CKS1 (FIG. 8). For example, NF10 induced LDH release at a concentration of 5 μM and had the highest activity among the engineered oncolytic peptides tested. All the peptides tested in FIG. 8 demonstrate cell-killing capability (except for NF22). The LDH assay quantifies the release of LDH as an indicator of cell death. As shown in FIGS. 9A and 9B the selected peptides cytotoxic capability.

The peptides can be further investigated for comparison to CKS1 by examining cell viability, cell morphology, LDH release in various cell types, and release of DAMPs following peptides treatments, as well as intratumoral injections of the peptides, as described above.

TABLE 1

| Compound | | SEQ |
|---|---|---|
| name | Sequence | ID NO: |
| CKS1 | NGRKACLNPASPIVKKIIEKMLNS | 2 |
| NF04 | NGRKACLNPASPIVKKIIKKMLNS | 3 |
| NF05 | NGRKACLNPASPIVKKIIKKILNS | 4 |
| NF06 | NGRKACLNPASPIVKKIIKKILKS | 5 |
| NF07 | NGRKACLNPASPIVKKIIKKILKI | 6 |
| NF08 | NGRKACLNPASPKVKKIIKKIKKI | 7 |
| NF09 | NGRKACLNPASIVKKIIKKILNS | 8 |
| NF10 | NGRKACLNPASIVKKIIKKILKS | 9 |
| NF11 | NGRKACLNPASIVKKIIKKILKI | 10 |
| NF12 | NGRKACLNPASKVKKIIKKIKKI | 11 |
| NF13 | NGRKACLNPAKKVKKIIKKIKKI | 12 |
| NF14 | NGRKAKKNPASPIVKKIIEKMLNS | 13 |
| NF15 | NGRKAKKNPASPIVKKIIKKILKI | 14 |
| NF16 | NGRKAKKNPASPKVKKIIKKIKKI | 15 |
| NF17 | NGRKAKKNPASIVKKIIKKILKI | 16 |
| NF18 | NGRKAKKNPASKVKKIIKKIKKI | 17 |
| NF19 | NGRKAKKNPAKKVKKIIKKIKKI | 18 |
| NF20 | NGRKACLNASIVKKIIKKILNS | 19 |
| NF21 | NGRKACLNAKKVKKIIKKIKKI | 20 |
| NF22 | NGRKAKKNAKKVKKIIKKIKKI | 21 |
| NF23 | NGRKACLNPASPIVRRIIERMLNS | 22 |
| NF24 | NGRKACLNPASPIVRRIIRRILRI | 23 |
| NF25 | NGRKACLNPARRVRRIIRRIRRI | 24 |
| NF26 | NGRKAKKNPARRVRRIIRRIRRI | 25 |
| NF27 | NGRKAKLNPASIVKKIIKKILKS | 26 |
| NF28 | NGRKAALNPASIVKKIIKKILKS | 27 |
| NF29 | NGRKALLNPASIVKKIIKKILKS | 28 |
| NF30 | NGRKANLNPASIVKKIIKKILKS | 29 |
| NF31 | NGRKAKLNPASFFKKIIKKILKS | 30 |
| NF32 | NGRKAKLNPASIVKKFFKKILKS | 31 |
| NF33 | NGRKAKLNPASIVKKIIKKFFKS | 32 |
| NF34 | NGRKAKLNPASFFKKFFKKILKS | 33 |
| NF35 | NGRKAKLNPASIVKKFFKKFFKS | 34 |
| NF36 | NGRKAKLNPASFFKKIIKKFFKS | 35 |
| NF37 | NGRKAKLNPASFFKKFFKKFFKS | 36 |
| NF38 | ASIVKKIIKKILKS | 37 |
| NF39 | RKAKLNPASIVKKIIKKILKS | 38 |
| NF40 | AKLNPASIVKKIIKKILKS | 39 |
| NF41 | LNPASIVKKIIKKILKS | 40 |

### Example 4

### Cancer Cytotoxicity

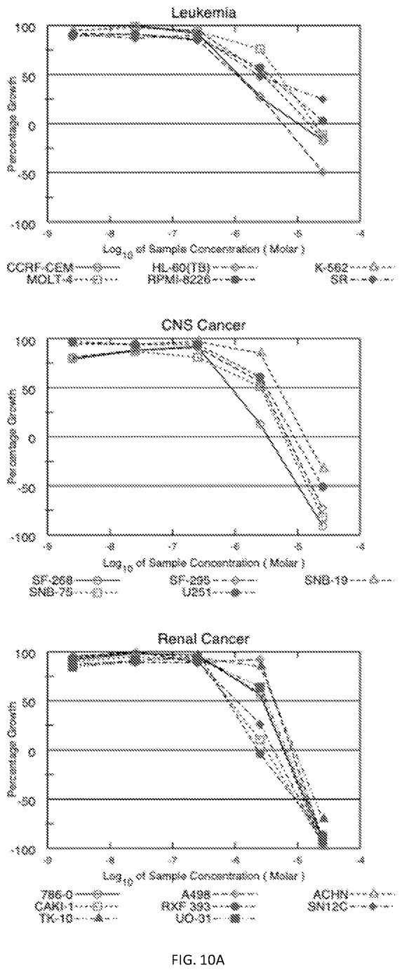

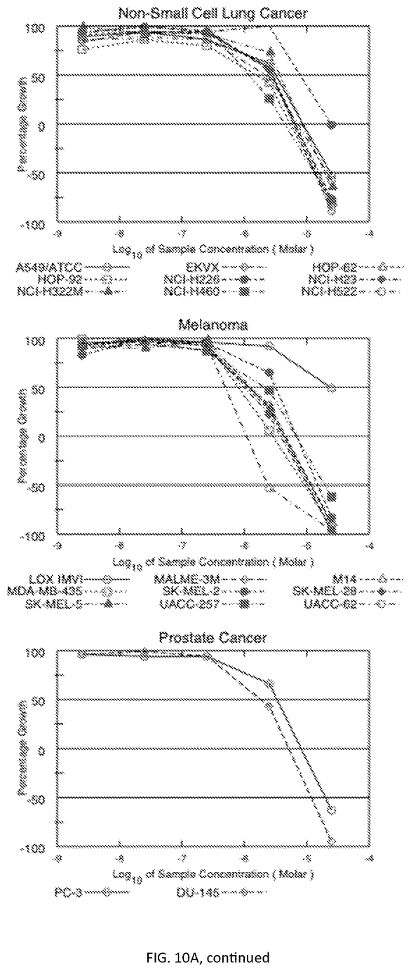

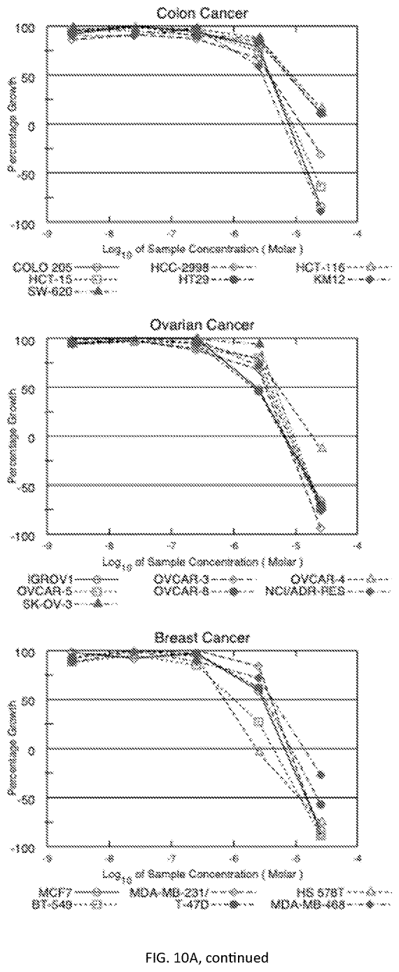

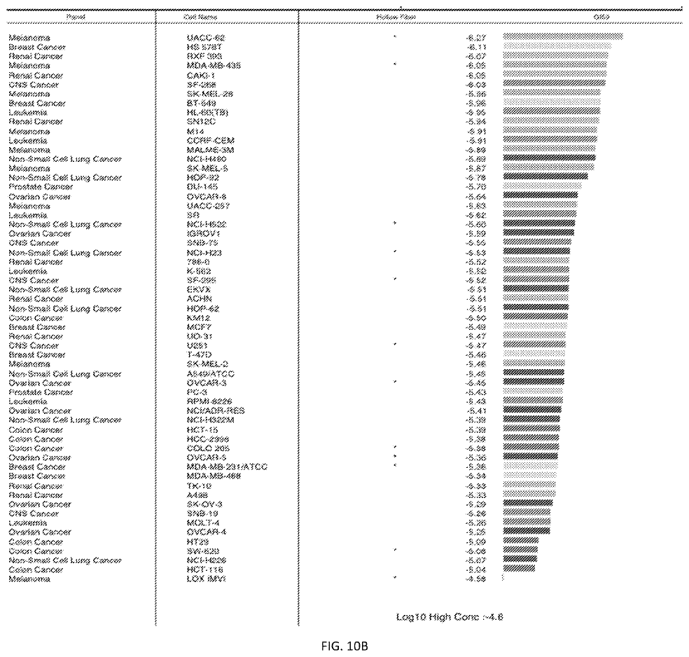

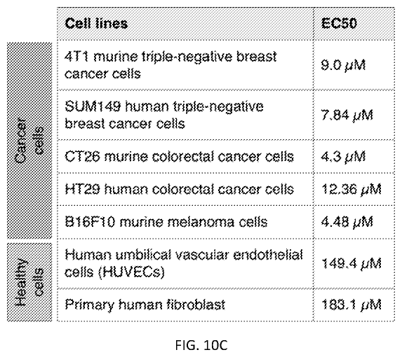

As shown in FIG. 10A, the cell viability of 59 different cell lines treated with 5 different concentrations of NF27 for 48 hours. The x-axis shows the concentration of NF27, and the y-axis shows the cell growth of cells treated with NF27. A value of 100% indicates that the cells grew as fast as the control (if NF27 did not affect the cell growth). A value of 0% indicates that the cells did not grow during the experiment (if NF27 completely inhibited cell growth). Negative values indicate that the number of cells decreased during the experiment. FIG. 10B summarizes the GI₅₀ (the concentration required to inhibit 50% of cell growth) of each cell line. Among the 59 cell lines, 58 cell lines demonstrated a GI₅₀ of less than 10 μM. LOX IMVI was significantly resistant to NF27 compared to the other cell lines, but still had a GI₅₀ of 26 μM. These data demonstrate that NF27 does not depend on specific markers expressed in specific cancer types and that NF27 is expected to show cytotoxicity in a wide range of cancer.

An LDH assay was used to demonstrate the cell-killing activity against cancer cells not included in the NCI-60 screen. The EC50 (the concentration required to kill 50% of the cells) is listed in FIG. 10C. Notably, NF27 showed significantly lower toxicity against healthy cells compared to cancer cells.

Additionally other cancer types, specifically lower rectal cancers and glioblastoma and metastasis in the liver can be utilized in similar experiments to test cytotoxic and anti-cancer effects of the peptides.

### Example 5

### Effects on Tumor Size

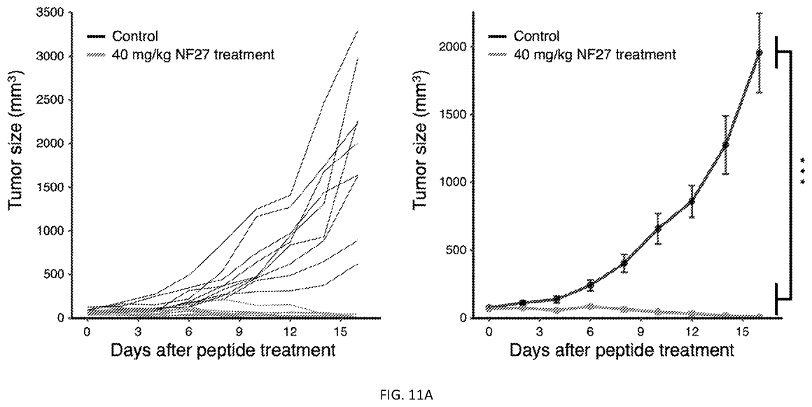

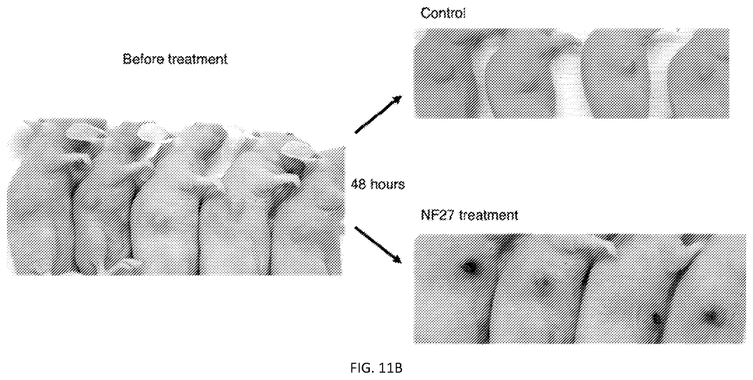

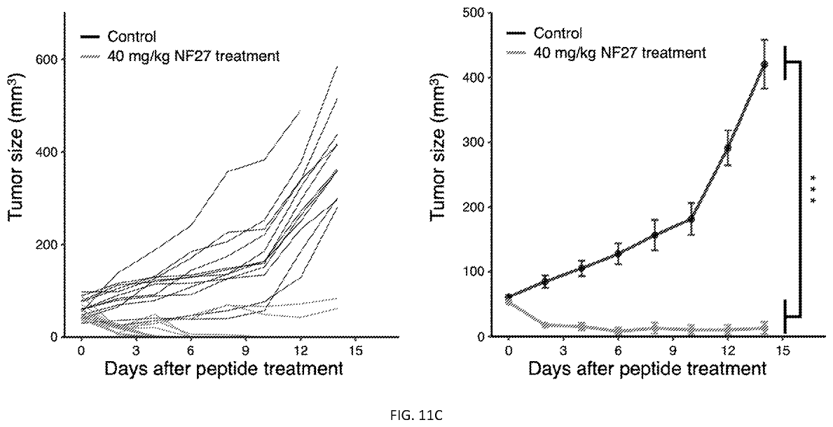

NF27 was used to test the effects against tumors established in the flanks of mice. CT26 murine colorectal tumors were established and treated daily with 40 mg/kg weight NF27. NF27 inhibited tumor growth in all tumors and eradicated the tumor in 6 out of 10 mice (FIG. 11A). HT29 human colorectal tumors were established in immunodeficient mice. NF27 (40 mg/kg weight) was administered intratumorally every other day. FIGS. 11B and 11C show the HT29 tumors going necrotic because of NF27 treatment. The growth of all tumors was inhibited, and 8 out of 10 tumors were eradicated during the treatment. Additionally, these studies used athymic, nude mice, suggesting that the cell-killing effects do not rely on T-cell mediated immune responses in vivo.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A peptide, or a pharmaceutically acceptable salt or solvate thereof, comprising an amino acid sequence of NGRKAX₁X₂NPAX₃PX₄ X₁₃X₅X₆ X₁₄X₁₅X₇X₈X₉X₁₀X₁₁X₁₂ (SEQ ID NO: 41), wherein:
X₁ is K, A, L, N or C;
X₂ is K or L;
X₃ is S, K, or R;
X₄ is I, R, K or F;
X₅, X₆, X₈ are each independently K or R;
X₇ is K, R, or E;
X₉ is M, I, or F;
X₁₀ is L, R, K, or F;
X₁₁ is N, R, or K;
X₁₂ is S or I;
X₁₃ is V or F;
X₁₄ and X₁₅ are each independently I or F; and
wherein the peptide has one or more amino acid substitutions, additions or deletions compared to NGRKACLNPASPIVKKIIEKMLNS (SEQ ID NO: 2).

2. The peptide of claim 1, wherein X₄ and X₁₃ are F.

3. The peptide of claim 1, wherein X₄ is I and X₁₃ is V.

4. The peptide of claim any of claims 1-3, wherein X₉ and X₁₀ are F.

5. The peptide of claim any of claims 1-3, wherein X₉ is I and X₁₀ is L.

6. The peptide of any of claims 1-5, wherein X₁₄ and X₁₅ are I.

7. The peptide of any of claims 1-5, wherein X₁₄ and X₁₅ are F.

8. The peptide of any of claims 1-7, wherein X₅, X₆, X₇, and X₈ are K.

9. The peptide of any of claims 1-8, wherein X₁₁ is K and X₁₂ is S.

10. The peptide of any of claim 1-9, wherein X₁ is K and X₂ is L.

11. The peptide of any of claims 1-10, wherein X₃ is S.

12. The peptide of any of claims 1-11, the proline at position 12 is absent.

13. A peptide of claim 1, comprising an amino acid sequence of NGRKAX₁X₂NPAX₃PX₄VX₅X₆IIX₇X₈X₉X₁₀X₁₁X₁₂ (SEQ ID NO: 1), wherein:
X₁ is K, A, L, N or C;
X₂ is K or L;
X₃ is S, K, or R;
X₄ is I, R or K;
X₅, X₆, X₈ are each independently K or R;
X₇ is K, R, or E;
X₉ is M or I;
X₁₀ is L, R, or K;
X₁₁ is N, R, or K;
X₁₂ is S or I; and
wherein the peptide has one or more amino acid substitutions, additions or deletions compared to NGRKACLNPASPIVKKIIEKMLNS (SEQ ID NO: 2).

14. The peptide of claim 13, wherein X₁ is C and X₂ is L.

15. The peptide of, claim 13 wherein X₁ and X₂ are K.

16. The peptide of any of claims 13-15, wherein X₃ is S.

17. The peptide of any of claims 13-16, wherein X₉ is I.

18. The peptide of any of claim 13-17, wherein X₁₂ is I.

19. The peptide of claim 17 18, wherein X₁₁ is K and X₁₂ is I.

20. The peptide of claim 18 or 19, wherein X₁₀ is K.

21. The peptide of any of claims 13-20, wherein X₅, X₆, and X₈ are R.

22. The peptide of claim 21, wherein X₇ and X₁₁ are R.

23. The peptide of claim 21 or 22, wherein X₉ and X₁₂ are I.

24. The peptide of any of claims 21-23, wherein X₁₀ is R.

25. The peptide of any of claims 21-24, wherein X₃ and X₄ are R.

26. The peptide of any of claims 13-20, wherein X₅, X₆, and X₈ are K.

27. The peptide of claim 26, wherein X₇ is K.

28. The peptide of claim 26 or 27, wherein X₉ is I and X₁₀ is L.

29. The peptide of any of claims 26-28, wherein X₁₁ is K and X₁₂ is I.

30. The peptide of any of claims 13-29, wherein the proline at position 9, the proline at position 12, or a combination thereof is absent.

31. The peptide of claim 30, wherein X₅, X₆, X₇, and X₈ are K.

32. The peptide of claim 30 or 31, wherein X₉ is I.

33. The peptide of any of claims 30-32, wherein X₁ is C and X₂ is L.

34. The peptide of any of claims 30-32, wherein X₁ is K, A, L, or N and X₂ is L.

35. The peptide of any of claims 30-32, wherein X₁ and X₂ are K.

36. The peptide of any of claims 30-35, wherein X₃ is S and X₄ is I.

37. The peptide of claim 36, wherein X₁₀ is L, X₁₁ is Nor K, and X₁₂ is S or I.

38. The peptide of any of claims 30-35, wherein X₃ and X₄ are K.

39. The peptide of claim 38, wherein X₁₀ and X₁₁ are K and X₁₂ is I.

40. The peptide of any of claims 30-39, wherein the prolines at positions 9 and 12 are absent and X₁ is C and X₂ is L.

41. The peptide of claim 40, wherein X₃ is S or A and X₄ is K or I.

42. The peptide of claim 40 or 41, wherein X₁₀ and X₁₁ are K, and X₁₂ is I.

43. The peptide of claim 40 or 41, wherein X₁₀ is L, X₁₁ is N, and X₁₂ is S.

44. The peptide of any of claim 1-43, wherein the amino acid sequence is alpha helical.

45. The peptide of any of claims 1-44, wherein the peptide has one or more amino acid substitutions, additions, or deletions compared to NGRKACLNPASIVKKIIKKILKS (SEQ ID NO: 9).

46. The peptide of any of claims 1-45, wherein the peptide comprises one of the following amino acid sequences: NGRKACLNPASPIVKKIIKKMLNS (SEQ ID NO: 3); NGRKACLNPASPIVKKIIKKILNS (SEQ ID NO: 4); NGRKACLNPASPIVKKIIKKILKS (SEQ ID NO: 5); NGRKACLNPASPIVKKIIKKILKI (SEQ ID NO: 6); NGRKACLNPASPKVKKIIKKIKKI (SEQ ID NO: 7); NGRKACLNPASIVKKIIKKILNS (SEQ ID NO: 8); NGRKACLNPASIVKKIIKKILKS (SEQ ID NO: 9); NGRKACLNPASIVKKIIKKILKI (SEQ ID NO: 10); NGRKACLNPASKVKKIIKKIKKI (SEQ ID NO: 11); NGRKACLNPAKKVKKIIKKIKKI (SEQ ID NO: 12); NGRKAKKNPASPIVKKIIEKMLNS (SEQ ID NO: 13); NGRKAKKNPASPIVKKIIKKILKI (SEQ ID NO: 14); NGRKAKKNPASPKVKKIIKKIKKI (SEQ ID NO: 15); NGRKAKKNPASIVKKIIKKILKI (SEQ ID NO: 16); NGRKAKKNPASKVKKIIKKIKKI (SEQ ID NO: 17); NGRKAKKNPAKKVKKIIKKIKKI (SEQ ID NO: 18); NGRKACLNASIVKKIIKKILNS (SEQ ID NO: 19); NGRKACLNAKKVKKIIKKIKKI (SEQ ID NO: 20); NGRKAKKNAKKVKKIIKKIKKI (SEQ ID NO: 21); NGRKACLNPASPIVRRIIERMLNS (SEQ ID NO: 22); NGRKACLNPASPIVRRIIRRILRI (SEQ ID NO: 23); NGRKACLNPARRVRRIIRRIRRI (SEQ ID NO: 24); NGRKAKKNPARRVRRIIRRIRRI (SEQ ID NO: 25); NGRKAKLNPASIVKKIIKKILKS (SEQ ID NO: 26); NGRKAALNPASIVKKIIKKILKS (SEQ ID NO: 27); NGRKALLNPASIVKKIIKKILKS (SEQ ID NO: 28); NGRKANLNPASIVKKIIKKILKS (SEQ ID NO: 29); NGRKAKLNPASFFKKIIKKILKS (SEQ ID NO: 30); NGRKAKLNPASIVKKFFKKILKS (SEQ ID NO: 31); NGRKAKLNPASIVKKIIKKFFKS (SEQ ID NO: 32); NGRKAKLNPASFFKKFFKKILKS (SEQ ID NO: 33); NGRKAKLNPASIVKKFFKKFFKS (SEQ ID NO: 34); NGRKAKLNPASFFKKIIKKFFKS (SEQ ID NO: 35); and NGRKAKLNPASFFKKFFKKFFKS (SEQ ID NO: 36).

47. A peptide, or a pharmaceutically acceptable salt or solvate thereof, comprising an amino acid sequence of ASIVKKIIKKILKS (SEQ ID NO: 37) and having less than 25 total amino acids.

48. The peptide of claim 47, wherein the peptide comprises one or more of the following amino acid sequences: NGRKACLNPASIVKKIIKKILKS (SEQ ID NO: 9); NGRKAKLNPASIVKKIIKKILKS (SEQ ID NO: 26); NGRKAALNPASIVKKIIKKILKS (SEQ ID NO: 27); NGRKALLNPASIVKKIIKKILKS (SEQ ID NO: 28); NGRKANLNPASIVKKIIKKILKS (SEQ ID NO: 29); ASIVKKIIKKILKS (SEQ ID NO: 37); RKAKLNPASIVKKIIKKILKS (SEQ ID NO: 38); AKLNPASIVKKIIKKILKS (SEQ ID NO: 39); and LNPASIVKKIIKKILKS (SEQ ID NO: 40).

49. A composition comprising a peptide of any of claims 1-48, or a nucleic acid encoding thereof.

50. The composition of claim 49, further comprising a carrier.

51. The composition of claim 49 or 50, wherein the carrier comprises water and the peptide is at a concentration of 1-100 mg/mL.

52. A method of lysing cancer cell membranes and/or and inducing cancer cell death comprising contacting a cancer cell with an effective amount of the peptide of any of claims 1-48, a nucleic acid encoding thereof, or the composition of any of claims 49-51.

53. The method of claim 52, wherein the cancer cell is in vitro or ex vivo.

54. The method of claim 52, wherein the cancer cell is in a subject.

55. The method of claim 54, wherein the contacting comprises administering to a subject in need thereof an effective amount of the peptide or a composition thereof.

56. A method of treating a disease or disorder comprising administering to a subject in need thereof an effective amount of the peptide of any of claims 1-48, a nucleic acid encoding thereof, or the composition of any of claims 49-51.

57. The method of claim 56, wherein the disease or disorder comprises cancer.

58. The method of claim 56 or 57, wherein the disease or disorder comprises breast cancer, skin cancer, and colorectal cancer.

59. The method of any of claims 56-58, wherein administering the peptide or composition thereof inhibits angiogenesis, ruptures cancer cell membranes, induces apoptotic pathways, induces cancer cell death, decreases tumor growth, and/or stimulates anti-cancer immunity.

60. The method of any of claims 54-59, wherein the subject is a human.

61. A peptide of any of claims 1-48, a nucleic acid encoding thereof, or a composition thereof for use in manufacturing a medicament for use in treating cancer.
